(19) United States
(12) Patent Application Publication
(10) Pub. No.: US 2016/0074380 A1
(43) Pub. Date: Mar. 17, 2016

(54) Treatment Of Neurodegenerative Diseases With Combination Of Laquinimod And Fingolimod

(71) Applicants: Michael Hayden, Petach-Tikva (IL); Liat Hayardeny, Tel Aviv (IL); David Ladkani, Jerusalem (IL)

(72) Inventors: Michael Hayden, Petach-Tikva (IL); Liat Hayardeny, Tel Aviv (IL); David Ladkani, Jerusalem (IL)

(73) Assignee: TEVA PHARMACEUTICAL INDUSTRIES LTD., Petach-Tikva (IL)

(21) Appl. No.: 14/854,790

(22) Filed: Sep. 15, 2015

Related U.S. Application Data
(60) Provisional application No. 62/050,842, filed on Sep. 16, 2014.

(51) Int. Cl. A61K 31/4704; A61K 45/06; A61K 31/137

(52) U.S. Cl. CPC A61K 31/4704; A61K 31/137; A61K 45/06

(57) ABSTRACT

This invention provides a method of treating a subject afflicted with a neurodegenerative disease comprising periodic administration of an amount of laquinimod and an amount of fingolimod, wherein the amounts when taken together are effective to treat the subject. Also provided are packages and pharmaceutical compositions comprising laquinimod and fingolimod for treating a subject afflicted with a neurodegenerative disease. Also provided is a pharmaceutical composition comprising laquinimod for use as an add-on therapy or in combination with fingolimod, and a pharmaceutical composition comprising fingolimod for use as an add-on therapy or in combination with laquinimod, for treating said subject.

This application claims benefit of U.S. Provisional Application No. 62/050,842, filed Sep. 16, 2014, the entire content of which is hereby incorporated by reference herein.

Throughout this application, various publications are referred to by first author and year of publication. Full citations for these publications are presented in a References section immediately before the claims. The disclosures of these documents and publications referred to herein are hereby incorporated in their entireties by reference into this application in order to more fully describe the state of the art to which this invention pertains.

### BACKGROUND

### Neurodegenerative Diseases

A neurodegenerative disease is an umbrella term for chronic degeneration of neurons in, e.g., the central nervous system (CNS), characterized by molecular and genetic changes in nerve cells that result in nerve cell degeneration and ultimately nerve dysfunction and death (Bertram, 2005). Neurodegenerative diseases include, but are not limited to, Alzheimer's disease (AD), Amyotrophic lateral sclerosis (ALS), Huntington's disease (HD), and Parkinson's disease (PD) (Chesselet, 2003; Hyman, 1991; Howell, 2000; Ciammola, 2007; Riviere, 1998; Katoh-Semba, 2002; and The Merck Manual).

### Alzheimer's Disease (AD)

Alzheimer's disease is characterized by a progressive inexorable loss of cognitive function. AD is characterized by two neuropathological hallmarks, excessive number of senile plaques in the cerebral cortex and subcortical gray matter, which also contains β-amyloid, and neurofibrillary tangles consisting of tau protein (Avila et al., 2011; and The Merck Manual). Senile plaques are extracellular deposits of amyloid fibrils composed of the β-amyloid peptide. NFT are intraneuronally generated aggregates of paired helical filaments (PHF), which are assembled from hyperphosphorylated forms of the microtubule-associated protein tau. Glycogen synthase kinase-3β(GSK3β) has been proposed as the link between these two neuropathological hallmarks and deregulation of GSK3β activity in neurons has been postulated as a key feature in AD pathogenesis based on the interaction of GSK3 with many of the cellular components related to the neuropathology of AD, such as the amyloid precursor protein, the β-amyloid peptide, the metabolic pathway leading to acetylcholine synthesis, the presenilins, which are mutated in many cases of familial AD, and tau protein (Avila et al., 2011).

### Amyotrophic Lateral Sclerosis (ALS)

Amyotrophic lateral sclerosis is a chronic and debilitating neurodegenerative disease which involves degeneration of cortical, bulbar and medullar motor neurons. Riluzole (2-amino-6-[trifluoromethoxy]benzothiazole) is an antagonist of glutamatergic neurotransmission that prolongs survival in ALS (Riviere, 1998). Riluzole has also been shown to significantly increase BDNF levels in the rat brain, thereby promoting precursor proliferation (Katoh-Semba, 2002).

### Huntington's Disease (HD)

Huntington's disease is a devastating inherited neurodegenerative disorder characterized by motor, cognitive, and psychiatric symptoms and by a progressive degeneration of neurons in basal ganglia in the brain cortex. Patients suffering from HD have significantly lower BDNF levels in serum compared to healthy controls (Ciammola, 2007; Phillips, 2009). The genetic defect of HD leads to a mutation in the ubiquitous protein, huntingtin, and neuronal loss, particularly in the caudate nucleus in early disease (Phillips, 2009).

### Parkinson's Disease (PD)

Parkinson's disease is a chronic and progressive degenerative disease of the brain that impairs motor control, speech, and other functions. One of the most striking features of Parkinson's disease is that it primarily affects a restricted neuronal population in the brain. Although other neurons are also affected, the dopaminergic neurons of the substantia nigra pars compacta are the most vulnerable to the disease process (Chesselet, 2003). BDNF has potent effects on survival and morphology of mesencephalic dopaminergic neurons, increasing their survival, and thus its loss could contribute to death of these cells in PD (Hyman, 1991; Howell, 2000).

### Multiple Sclerosis (MS)

Multiple sclerosis is known to be an autoimmune disease that affects the brain and spinal cord, which is assumed to be mediated by an autoimmune process possibly triggered by infection and superimposed upon a genetic predisposition. However, recently some have suggested that multiple sclerosis is not primarily an autoimmune disease but instead is due to a neurodegenerative process that sparks an inflammatory response (Anderson, 2013).

### Fingolimod

Fingolimod (Fingolimod, Gilenya™) is a new class of drugs called sphingosine 1-phosphate (S1P) receptor modulators. These medicines reduce inflammation and may also have a direct beneficial effect on cells in the central nervous system (CNS). Upon administration, fingolimod is phosphorylated by sphingosine kinase to form the active metabolite fingolimod-phosphate—Fingolimod is therefore a prodrug. Fingolimod-phosphate binds the sphingosine 1-phosphate receptors S1PR-1, S1PR3, S1PR4 and S1PR5 with high affinity and thereby blocks the capacity of leukocytes to migrate from lymph nodes into the peripheral blood. These receptors are also known as EDG receptors, and are all members of the rhodospin-like GPCR family, the largest single historical successful family of drug targets (GPCR SARfari: S1PR-1 (aka. EDG1)). The curative mechanism underlying fingolimod's therapeutic effect is unknown but may involve a reduced migration of lymphocytes into the CNS.

The chemical structure of fingolimod was derived from the myriocin (ISP-1) metabolite of the fungus Isaria sinclairii. It is a structural analogue of sphingosine and gets phosphorylated by sphingosine kinases in the cell (most importantly sphingosine kinase 2) (Paugh, 2003; Billich, 2003; Sanchez, 2003). The molecular biology of phospho-fingolimod is thought to lie in its activity at one of the five sphingosine-1-phosphate receptors, S1PR1 (Hla, 2001). It can sequester lymphocytes in lymph nodes, preventing them from moving to the central nervous system for auto-immune responses in multiple sclerosis and was originally proposed as an anti-rejection medication indicated post-transplantation. It has been reported to stimulate the repair process of glial cells and precursor cells after injury (Horga, 2008). Fingolimod has also been reported to be a cannabinoid receptor antagonist (Paugh S W, 2006), a cPLA2 inhibitor (Payne S G, 2007) and a ceramide synthase inhibitor (Berdyshev E V, 2009).

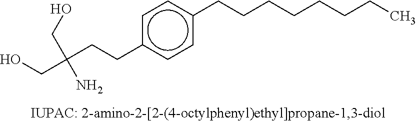

The approved medication Gilenya is an oral capsule containing 0.56 mg of the hydrochloride salt of fingolimod which is equivalent to 0.5 mg of fingolimod.

### Laquinimod

Laquinimod is a novel synthetic compound with high oral bioavailability which has been suggested as an oral formulation for the treatment of Multiple Sclerosis (MS) (Polman, 2005; Sandberg-Wollheim, 2005; Comi et al, 2007). Laquinimod and its sodium salt form are described, for example, in U.S. Pat. No. 6,077,851. The mechanism of action of laquinimod is not fully understood.

Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runstrom, 2006; Brück, 2011).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

### Combination Therapy

The administration of two drugs to treat a given condition, such as multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of fingolimod and interferon (IFN) has been experimentally shown to abrogate the clinical effectiveness of either therapy. (Brod, 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick, 2006; Kleinschmidt-DeMasters, 2005; Langer-Gould, 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of combination therapy of two drugs, in particular laquinimod and fingolimod, cannot be predicted until the results of a combination study are available.

### BRIEF DESCRIPTION OF THE DRAWINGS

FIG. 1 shows the effect of laquinimod on demyelination in the cuprizone model.

FIG. 2 depicts demyelination in lateral and medial corpus callosum.

FIG. 3 shows the effect of laquinimod on remyelination in the cuprizone model.

FIG. 4 shows effect of laquinimod on lysolecithin-induced demyelination in the lysolecithin model.

FIG. 5 shows the effect of laquinimod on established EAE.

FIG. 6 shows the effect of laquinimod on established EAE.

FIG. 7 shows the effect of laquinimod and FTY 720 on oligodendrocyte survival.

FIG. 8 shows the effect of laquinimod on oxidative glutamate toxicity of H2TT (primary neuronal culture) cells.

FIG. 9 shows the effect of laquinimod on human astrocyte activation.

FIG. 10 shows the effect of laquinimod on human astrocyte activation.

FIG. 11 shows the effect of laquinimod on the regulation of pro-inflammatory cytokine secretion from human astrocytes in vitro.

FIG. 12 shows the effect of laquinimod on p65 translocation into the astrocyte nucleus in vivo.

FIG. 13 shows the effect of laquinimod on microglial activation in culture.

FIG. 14 shows the effect of laquinimod on inhibition of microglial production of pro-inflammatory cytokine in human microglia.

FIG. 15 shows the effect of laquinimod on inhibition of microglial activation in EAE-afflicted mice.

FIG. 16 shows the effect of laquinimod on lymphocyte counts.

FIG. 17 shows the effect of laquinimod in the penetration of both intact and disrupted Blood Brain Barrier (BBB).

FIG. 18 shows the effect of fingolimod (FTY 720) on re-myelination in the cuprizone model.

FIG. 19 shows the effect of FTY 720 on re-myelination in the lysolecithin-induced demyelination model.

FIG. 20 shows the effect of S1P, FTY 720, and FTY 720-P in the pretreatment of mouse-cultured cortical cells.

FIG. 21 shows the effect of FTY 720 on inhibition of microglial production of pro-inflammatory cytokine in mouse primary microglia.

FIG. 22 shows the effect of fingolimod dosage on the reduction of peripheral lymphocyte counts.

FIG. 23 shows the high brain/plasma ratio of fingolimod in Dark Agouti (DA) experimental autoimmune encephalomyelitis (EAE) induced rats.

FIG. 24 shows the effect of laquinimod and FTY 720 on astrocytic and microglial activation and acute axonal damage.

FIG. 25 shows the effect of laquinimod and FTY 720 on chronic EAE.

FIG. 26 shows the effect of the co-administration of laquinimod and fingolimod in chronic EAE mice.

FIG. 27 shows the drug-drug interaction effects in the co-administration of laquinimod and fingolimod, as analyzed through pharmacokinetic (PK) attributes, such as levels, half-life and ADC.

FIG. 28 shows the effect of Laquinimod and Fingolimod treatment on NO release (pg/mL) in conditioned media from reactive astrocytes. Data are expressed in pg/mL (mean±sem; * p<0.05; ** p<0.01; *** p<0.001; one way Anova followed by Dunnett's test). # represents the condition of intoxication, p<0.001.

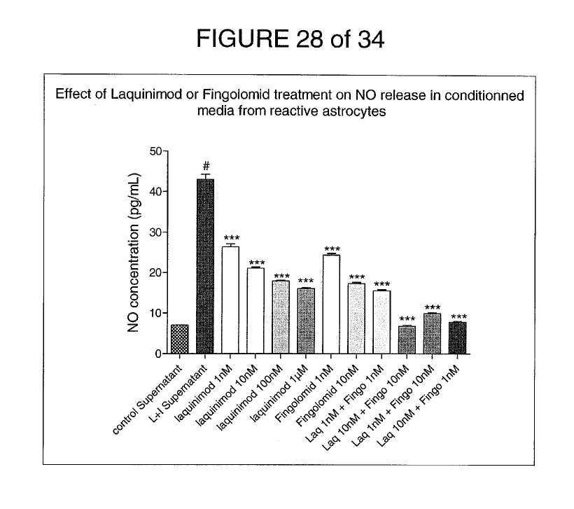

FIG. 29 shows the effect of Laquinimod and Fingolimod treatment on CCL7 release (pg/mL) in conditioned media from reactive astrocytes. Data are expressed in pg/mL (mean±sem; * p<0.05; ** p<0.01; *** p<0.001; one way Anova followed by Dunnett's test). # represents the condition of intoxication, p<0.001.

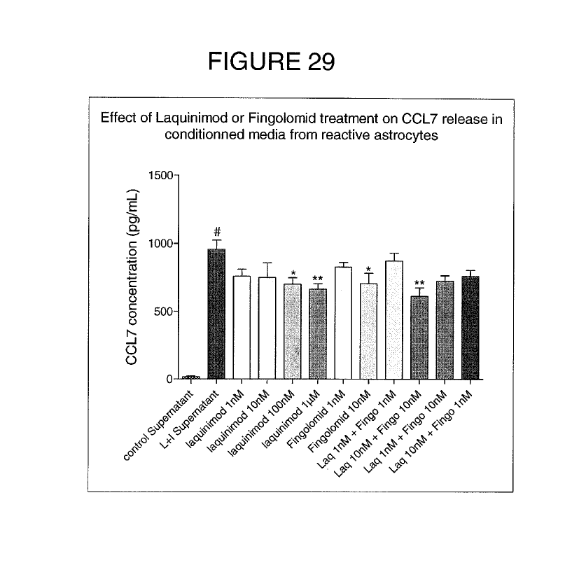

FIG. 30 shows the evaluation of IL-6 concentration (pg/mL) in conditioned media from reactive astrocytes after treatment with Laquinimod and Fingolimod by cytometry. Data are expressed in pg/mL (mean±sem; * p<0.05; ** p<0.01; *** p<0.001; one way Anova followed by Dunnett's test). # represents the condition of intoxication, p<0.001.

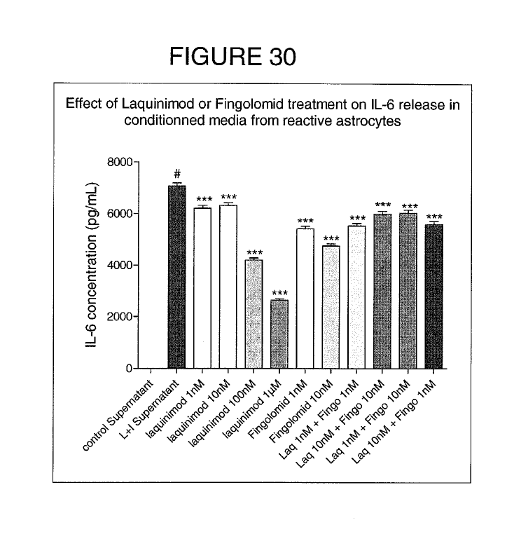

FIG. 31 shows the evaluation of IL-12-p70 concentration (pg/mL) in conditioned media from reactive astrocytes after treatment with Laquinimod and Fingolimod by cytometry. Data are expressed in pg/mL (mean±sem; * p<0.05; ** p<0.01; *** p<0.001; one way Anova followed by Dunnett's test). # represents the condition of intoxication, p<0.001.

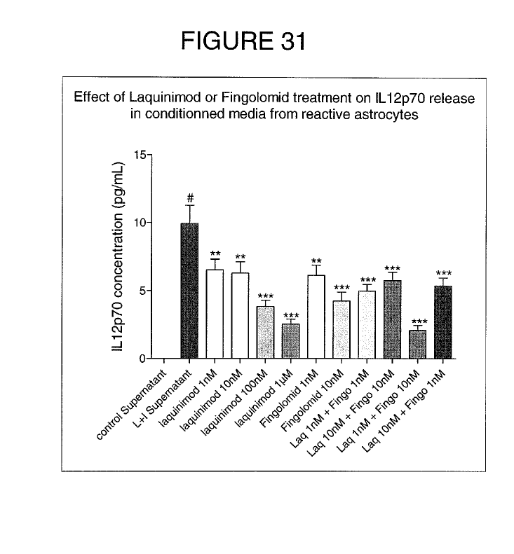

FIG. 32 shows the evaluation of TNFα concentration (pg/mL) in conditioned media from reactive astrocytes after treatment with Laquinimod and Fingolimod by cytometry. Data are expressed in pg/mL (mean±sem; * p<0.05; ** p<0.01; *** p<0.001; one way Anova followed by Dunnett's test). # represents the condition of intoxication, p<0.001.

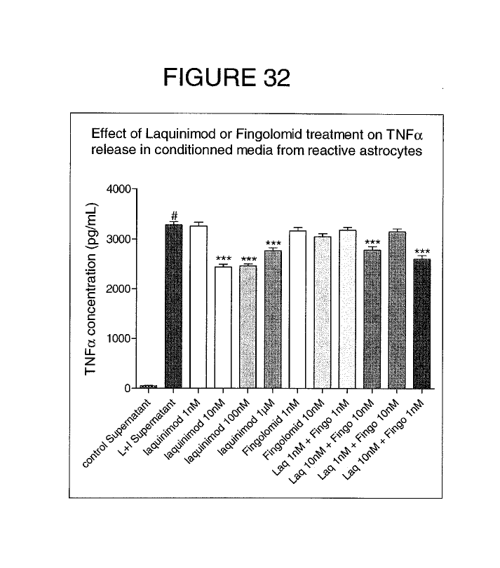

FIG. 33 shows the evaluation of GM-CSF concentration (pg/mL) in conditioned media from reactive astrocytes after treatment with Laquinimod and Fingolimod by cytometry. Data are expressed in pg/mL (mean±sem; * p<0.05; ** p<0.01; *** p<0.001; one way Anova followed by Dunnett's test). # represents the condition of intoxication, p<0.001.

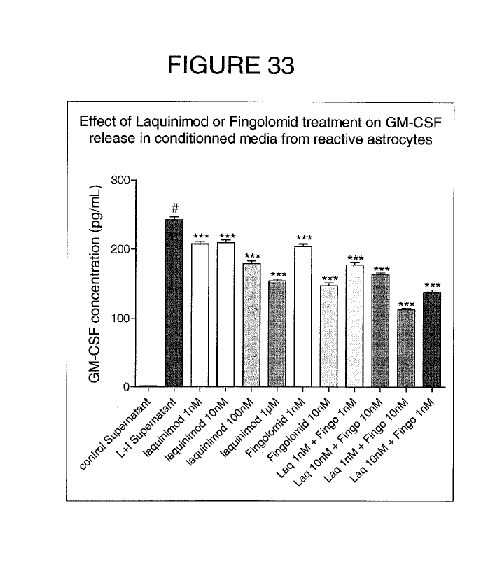

FIG. 34 shows the effect of a 72H treatment with conditioned media from reactive astrocytes (6 hr with LPS 100 ng/mL+IFNγ 10 ng/mL) in presence or not of Laguinimod on cortical neuron survival. Data are expressed in percentage of control (mean±sem; * p<0.05; ** p<0.01; *** p<0.001). Statistical analyses were performed using GraphPad Prism using unpaired one way Anova followed by Dunnett's test). # represents the condition of intoxication.

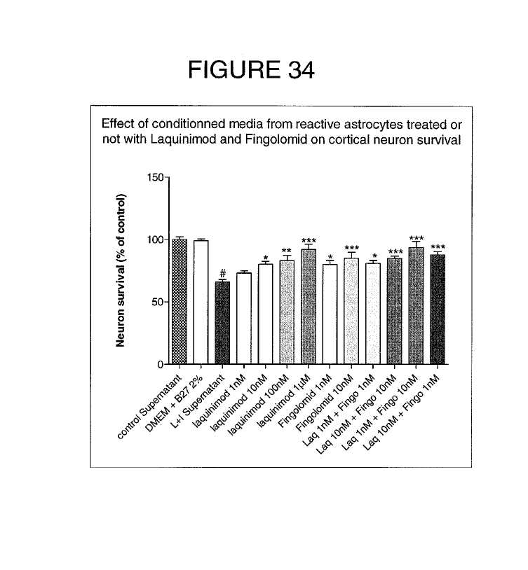

### SUMMARY OF THE INVENTION

This invention provides a method of treating a subject afflicted with a neurodegenerative disease comprising periodically administering to the subject an amount of laquinimod and an amount of fingolimod, wherein the amounts when taken together are effective to treat the subject.

This invention also provides a package comprising: a) a first pharmaceutical composition comprising an amount of laquinimod and a pharmaceutically acceptable carrier; b) a second pharmaceutical composition comprising an amount of fingolimod and a pharmaceutically acceptable carrier; and c) instructions for use of the first and second pharmaceutical compositions together to treat a subject afflicted with a neurodegenerative disease.

This invention also provides laquinimod for use as an add-on therapy or in combination with fingolimod or in treating a subject afflicted with a neurodegenerative disease.

This invention also provides a pharmaceutical composition comprising an amount of laquinimod and an amount of fingolimod for use in treating a subject afflicted with a neurodegenerative disease, wherein the laquinimod and the fingolimod are administered simultaneously, contemporaneously or concomitantly.

This invention also provides use of an amount of laquinimod and an amount of fingolimod in the preparation of a combination for treating a subject afflicted with a neurodegenerative disease wherein the laquinimod or pharmaceutically acceptable salt thereof and the fingolimod or pharmaceutically acceptable salt thereof are administered simultaneously, contemporaneously or concomitantly.

This invention also provides a pharmaceutical composition comprising an amount of laquinimod for use in treating a subject afflicted with a neurodegenerative disease as an add-on therapy or in combination with fingolimod by periodically administering the pharmaceutical composition and the fingolimod to the subject.

This invention also provides a pharmaceutical composition comprising an amount of fingolimod for use treating a subject afflicted with a neurodegenerative disease as an add-on therapy or in combination with laquinimod by periodically administering the pharmaceutical composition and the laquinimod to the subject.

This invention also provides a therapeutic package for dispensing to, or for use in dispensing to, a subject afflicted with a neurodegenerative disease, which comprises: a) one or more unit doses, each such unit dose comprising: i) an amount of laquinimod and ii) an amount of fingolimod wherein the respective amounts of said laquinimod and said fingolimod in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.

This invention also provides a pharmaceutical composition in unit dosage form, useful in treating a subject afflicted with a neurodegenerative disease, which comprises: a) an amount of laquinimod; b) an amount of fingolimod, wherein the respective amounts of said laquinimod and said fingolimod in said composition are effective, upon concomitant administration to said subject of one or more of said unit dosage forms of said composition, to treat the subject.

### DETAILED DESCRIPTION OF THE INVENTION

This invention provides a method of treating a subject afflicted with a neurodegenerative disease comprising periodically administering to the subject an amount of laquinimod and an amount of fingolimod, wherein the amounts when taken together are effective to treat the subject.

This invention also provides a method of treating a human patient afflicted with a neurodegenerative disease comprising periodically administering to the patient an amount of laquinimod and an amount of fingolimod, wherein the amounts when taken together is more effective to treat the human patient than when each agent is administered alone.

In an embodiment, the amount of laquinimod and the amount of fingolimod when administered together is more effective to treat the subject than when each agent at the same amount is administered alone.

In one embodiment, the neurodegenerative disease is other than a form of multiple sclerosis. In another embodiment, the neurodegenerative disease is Alzheimer's disease, Amyotrophic lateral sclerosis, Huntington's disease or Parkinson's disease.

In another embodiment, the neurodegenerative disease is Alexander disease, cerebellar ataxia, spinocerecellar ataxia (SCA), Batten disease, Creutzfeldt-Jakob disease, Charcot-Marie-Tooth disease (CMT), HIV-associated dementia, multiple system atrophy (MSA) or prion-related disease.

In one embodiment, the neurodegenerative disease is a Central Nervous System (CNS) Degenerative disease. In another embodiment, the neurodegenerative disease is a Peripheral Nervous System (PNS) Degenerative disease.

In an embodiment, the amount of laquinimod and the amount of fingolimod when taken together are effective to reduce or alleviate a symptom of the neurodegenerative disease in the subject. In a first embodiment, where the disease is Alzheimer's disease, the symptom is dementia, memory loss, cognitive impairment, personality change, psychiatric disorder, or functional impairment. In a second embodiment, where the disease is Amyotrophic lateral sclerosis, the symptom is cognitive impairment, motor function impairment, muscle disorder, fatigue, or functional impairment. In a third embodiment, where the disease is Huntington's disease, the symptom is memory loss, psychiatric disorder, cognitive impairment, motor function impairment, chorea, seizure, or functional impairment. In a fourth embodiment, where the disease is Parkinson's disease, the symptom is dementia, bradyphrenia, psychiatric disorder, cognitive impairment, motor function impairment, tremor, rigidity, bradykinesia, postural dysfunction, or functional impairment.

In one embodiment, the amount of laquinimod and the amount of fingolimod when taken together are effective to reduce cellular production of pro-inflammatory mediator. In one embodiment, the pro-inflammatory mediator is nitric oxide (NO). In another embodiment, the pro-inflammatory mediator is a cytokine. In one embodiment, the cytokine is chemokine (C-C motif) ligand 7 (CCL-7). In one embodiment, the cytokine is interleukin-6 (IL-6). In one embodiment, the cytokine is interleukin-12p70 (IL-12p70). In one embodiment, the cytokine is tumor necrosis factor alpha (TNF-α). In one embodiment, the cytokine is granulocyte-macrophage colony-stimulating factor (GM-CSF).

In one embodiment, the amount of laquinimod and the amount of fingolimod when taken together are effective to increase neuron survival. In one embodiment, the amount of laquinimod and the amount of fingolimod when taken together are effective to decrease neuron death. In one embodiment, the neuron is cortical neuron.

In one embodiment, laquinimod is laquinimod sodium. In another embodiment, fingolimod is fingolimod hydrochloride.

In one embodiment, the laquinimod and/or the fingolimod is administered via oral administration. In another embodiment, the laquinimod and/or the fingolimod is administered daily. In another embodiment, the laquinimod and/or the fingolimod is administered more often than once daily. In another embodiment, the laquinimod and/or the fingolimod is administered less often than once daily.

In one embodiment, the amount laquinimod administered is less than 0.6 mg/day. In another embodiment, the amount laquinimod administered is 0.1-40.0 mg/day. In another embodiment, the amount laquinimod administered is 0.1-2.5 mg/day. In another embodiment, the amount laquinimod administered is 0.25-2.0 mg/day. In another embodiment, the amount laquinimod administered is 0.5-1.2 mg/day. In another embodiment, the amount laquinimod administered is 0.25 mg/day. In another embodiment, the amount laquinimod administered is 0.3 mg/day. In another embodiment, the amount laquinimod administered is 0.5 mg/day. In another embodiment, the amount laquinimod administered is 0.6 mg/day.

In one embodiment, the amount of fingolimod administered is less than 0.5 mg/day. In another embodiment, the amount of fingolimod administered is 0.01-2.5 mg/day. In another embodiment, the amount of fingolimod administered is 2.5 mg/day. In another embodiment, the amount of fingolimod administered is 0.01-1 mg/day. In another embodiment, the amount of fingolimod administered is 0.1 mg/day. In another embodiment, the amount of fingolimod administered is 0.25 mg/day. In another embodiment, the amount of fingolimod administered is 0.5 mg/day.

In one embodiment, a loading dose of an amount different from the intended dose is administered for a period of time at the start of the periodic administration. In another embodiment, the loading dose is double the amount of the intended dose.

In one embodiment, the subject is receiving laquinimod therapy prior to initiating fingolimod therapy. In another embodiment, the administration of laquinimod substantially precedes the administration of fingolimod. In one embodiment, the subject is receiving fingolimod therapy prior to initiating laquinimod therapy. In another embodiment, the administration of fingolimod substantially precedes the administration of laquinimod. In another embodiment, the subject is receiving fingolimod therapy for at least 24 weeks prior to initiating laquinimod therapy. In another embodiment, the subject is receiving fingolimod therapy for at least 28 weeks prior to initiating laquinimod therapy. In another embodiment, the subject is receiving fingolimod therapy for at least 48 weeks prior to initiating laquinimod therapy. In yet another embodiment, the subject is receiving fingolimod therapy for at least 52 weeks prior to initiating laquinimod therapy.

In one embodiment, the method further comprises administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

In one embodiment, the periodic administration of laquinimod and fingolimod continues for at least 3 days. In another embodiment, the periodic administration of laquinimod and fingolimod continues for more than 30 days. In another embodiment, the periodic administration of laquinimod and fingolimod continues for more than 42 days. In another embodiment, the periodic administration of laquinimod and fingolimod continues for 8 weeks or more. In another embodiment, the periodic administration of laquinimod and fingolimod continues for at least 12 weeks. In another embodiment, the periodic administration of laquinimod and fingolimod continues for at least 24 weeks. In another embodiment, the periodic administration of laquinimod and fingolimod continues for more than 24 weeks. In yet another embodiment, the periodic administration of laquinimod and fingolimod continues for 6 months or more.

In one embodiment, each of the amount of laquinimod when taken alone, and the amount of fingolimod when taken alone is effective to treat the subject. In another embodiment, either the amount of laquinimod when taken alone, the amount of fingolimod when taken alone, or each such amount when taken alone is not effective to treat the subject. In yet another embodiment, the subject is a human patient.

This invention also provides a package comprising: a) a first pharmaceutical composition comprising an amount of laquinimod and a pharmaceutically acceptable carrier; b) a second pharmaceutical composition comprising an amount of fingolimod and a pharmaceutically acceptable carrier; and c) instructions for use of the first and second pharmaceutical compositions together to treat a subject afflicted with a neurodegenerative disease.

In one embodiment, the neurodegenerative disease is other than a form of multiple sclerosis. In another embodiment, the neurodegenerative disease is Alzheimer's disease, Amyotrophic lateral sclerosis, Huntington's disease or Parkinson's disease. In another embodiment, the neurodegenerative disease is Alexander disease, cerebellar ataxia, spinocerecellar ataxia (SCA), Batten disease, Creutzfeldt-Jakob disease, Charcot-Marie-Tooth disease (CMT), HIV-associated dementia, multiple system atrophy (MSA) or prion-related disease.

In one embodiment, the neurodegenerative disease is a Central Nervous System (CNS) Degenerative disease. In another embodiment, the neurodegenerative disease is a Peripheral Nervous System (PNS) Degenerative disease.

In one embodiment, the first pharmaceutical composition, the second pharmaceutical composition, or both the first and the second pharmaceutical composition are in an aerosol, an inhalable powder, an injectable a liquid, a solid, a capsule or a tablet form. In one embodiment, the first pharmaceutical composition, the second pharmaceutical composition, or both the first and the second pharmaceutical composition are in liquid form. In another embodiment, the first pharmaceutical composition, the second pharmaceutical composition, or both the first and the second pharmaceutical composition are in solid form. In another embodiment, the first pharmaceutical composition, the second pharmaceutical composition, or both the first and the second pharmaceutical composition are in capsule form. In another embodiment, the first pharmaceutical composition, the second pharmaceutical composition, or both the first and the second pharmaceutical composition are in tablet form. In another embodiment, the tablets are coated with a coating which inhibits oxygen from contacting the core. In another embodiment, the coating comprises a cellulosic polymer, a detackifier, a gloss enhancer, or pigment.

In one embodiment, the first pharmaceutical composition further comprises mannitol. In another embodiment, the first pharmaceutical composition further comprises an alkalinizing agent. In another embodiment, the alkalinizing agent is meglumine.

In one embodiment, the first pharmaceutical composition further comprises an oxidation reducing agent. In another embodiment, the first pharmaceutical composition is stable and free of an alkalinizing agent or an oxidation reducing agent. In another embodiment, the first pharmaceutical composition is free of an alkalinizing agent and free of an oxidation reducing agent. In another embodiment, the first pharmaceutical composition is stable and free of disintegrant.

In one embodiment, the first pharmaceutical composition further comprises a lubricant. In another embodiment, the lubricant is present in the composition as solid particles. In another embodiment, the lubricant is sodium stearyl fumarate or magnesium stearate.

In one embodiment, the first pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In yet another embodiment, the filler is mannitol or lactose monohydrate.

In an embodiment, the package further comprises a desiccant. In another embodiment, the desiccant is silica gel.

In one embodiment, the first pharmaceutical composition is stable and has a moisture content of no more than 4%. In another embodiment, laquinimod is present in the composition as solid particles. In another embodiment, the package is a sealed packaging having a moisture permeability of not more than 15 mg/day per liter. In another embodiment, the sealed package is a blister pack in which the maximum moisture permeability is no more than 0.005 mg/day. In another embodiment, the sealed package is a bottle. In another embodiment, the bottle is closed with a heat induction liner. In another embodiment, the sealed package comprises an HDPE bottle. In another embodiment, the sealed package comprises an oxygen absorbing agent. In yet another embodiment, the oxygen absorbing agent is iron.

In an embodiment of the present invention, the amount of laquinimod in the first composition is less than 0.6 mg. In another embodiment, the amount of laquinimod in the first composition is 0.1-40.0 mg. In another embodiment, the amount of laquinimod in the first composition is 0.1-2.5 mg. In another embodiment, the amount of laquinimod in the first composition is 0.25-2.0 mg. In another embodiment, the amount of laquinimod in the first composition is 0.5-1.2 mg. In another embodiment, the amount of laquinimod in the first composition is 0.25 mg. In another embodiment, the amount of laquinimod in the first composition is 0.3 mg. In another embodiment, the amount of laquinimod in the first composition is 0.5 mg. In another embodiment, the amount of laquinimod in the first composition is 0.6 mg.

In an embodiment of the present invention, the amount of fingolimod in the second composition is less than 0.5 mg. In another embodiment of the present invention, the amount of fingolimod in the second composition is 0.01-2.5 mg. In another embodiment, the amount of fingolimod in the second composition is 2.5 mg. In another embodiment, the amount of fingolimod in the second composition is 0.01-1 mg. In another embodiment, the amount of fingolimod in the second composition is 0.1 mg. In another embodiment, the amount of fingolimod in the second composition is 0.25 mg. In another embodiment, the amount of fingolimod in the second composition is 0.5 mg.

This invention also provides laquinimod for use as an add-on therapy or in combination with fingolimod or in treating a subject afflicted with a neurodegenerative disease.

This invention also provides a pharmaceutical composition comprising an amount of laquinimod and an amount of fingolimod for use in treating a subject afflicted with a neurodegenerative disease, wherein the laquinimod and the fingolimod are administered simultaneously, contemporaneously or concomitantly.

In one embodiment, the neurodegenerative disease is other than a form of multiple sclerosis. In another embodiment, the neurodegenerative disease is Alzheimer's disease, Amyotrophic lateral sclerosis, Huntington's disease or Parkinson's disease. In another embodiment, the neurodegenerative disease is Alexander disease, cerebellar ataxia, spinocerecellar ataxia (SCA), Batten disease, Creutzfeldt-Jakob disease, Charcot-Marie-Tooth disease (CMT), HIV-associated dementia, multiple system atrophy (MSA) or prion-related disease.

In one embodiment, the neurodegenerative disease is a Central Nervous System (CNS) Degenerative disease. In another embodiment, the neurodegenerative disease is a Peripheral Nervous System (PNS) Degenerative disease.

This invention also provides a pharmaceutical composition comprising an amount of laquinimod and an amount of fingolimod.

In one embodiment, laquinimod is laquinimod sodium. In another embodiment, fingolimod is fingolimod hydrochloride.

In one embodiment, the composition is in an aerosol, an inhalable powder, an injectable, a liquid, a solid, a capsule or a tablet form. In another embodiment, the composition is in liquid form. In another embodiment, the composition is in solid form. In another embodiment, the composition is in capsule form. In another embodiment, the composition is in tablet form.

In one embodiment, the tablets are coated with a coating which inhibits oxygen from contacting the core. In another embodiment, the coating comprises a cellulosic polymer, a detackifier, a gloss enhancer, or pigment.

In one embodiment, the pharmaceutical composition further comprises mannitol. In another embodiment, the pharmaceutical composition further comprises an alkalinizing agent. In another embodiment, the alkalinizing agent is meglumine. In an embodiment, the pharmaceutical composition comprises an oxidation reducing agent.

In an embodiment the pharmaceutical composition is free of an alkalinizing agent or an oxidation reducing agent. In another embodiment, the pharmaceutical composition is free of an alkalinizing agent and free of an oxidation reducing agent.

In one embodiment, the pharmaceutical composition is stable and free of disintegrant. In another embodiment, the pharmaceutical composition further comprises a lubricant. In another embodiment, the lubricant is present in the composition as solid particles. In another embodiment, the lubricant is sodium stearyl fumarate or magnesium stearate.

In an embodiment, the pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In another embodiment, the filler is mannitol or lactose monohydrate.

In one embodiment, the amount of laquinimod in the composition is less than 0.6 mg. In another embodiment, the amount of laquinimod in the composition is 0.1-40.0 mg. In another embodiment, the amount of laquinimod in the composition is 0.1-2.5 mg. In another embodiment, the amount of laquinimod in the composition is 0.25-2.0 mg. In another embodiment, the amount of laquinimod in the composition is 0.1-2.5 mg. In another embodiment, the amount of laquinimod in the composition is 0.25 mg. In another embodiment, the amount of laquinimod in the composition is 0.3 mg. In another embodiment, the amount of laquinimod in the composition is 0.5 mg. In another embodiment, the amount of laquinimod in the composition is 0.6 mg.

In one embodiment, the amount of fingolimod in the composition is less than 0.5 mg. In another embodiment, the amount of fingolimod in the composition is 0.01-2.5 mg. In another embodiment, the amount of fingolimod in the composition is 2.5 mg. In another embodiment, the amount of fingolimod in the composition is 0.01-1 mg. In another embodiment, the amount of fingolimod in the composition is 0.1 mg. In another embodiment, the amount of fingolimod in the composition is 0.25 mg. In another embodiment, the amount of fingolimod in the composition is 0.5 mg.

This invention also provides use of an amount of laquinimod and an amount of fingolimod in the preparation of a combination for treating a subject afflicted with a neurodegenerative disease wherein the laquinimod or pharmaceutically acceptable salt thereof and the fingolimod or pharmaceutically acceptable salt thereof are administered simultaneously, contemporaneously or concomitantly.

This invention also provides a pharmaceutical composition comprising an amount of laquinimod for use in treating a subject afflicted with a neurodegenerative disease as an add-on therapy or in combination with fingolimod by periodically administering the pharmaceutical composition and the fingolimod to the subject.

This invention also provides a pharmaceutical composition comprising an amount of fingolimod for use treating a subject afflicted with a neurodegenerative disease as an add-on therapy or in combination with laquinimod by periodically administering the pharmaceutical composition and the laquinimod to the subject.

This invention also provides a therapeutic package for dispensing to, or for use in dispensing to, a subject afflicted with a neurodegenerative disease, which comprises: a) one or more unit doses, each such unit dose comprising: i) an amount of laquinimod and ii) an amount of fingolimod wherein the respective amounts of said laquinimod and said fingolimod in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject. In an embodiment, the respective amounts of said laquinimod and said fingolimod in said unit dose when taken together is more effective to treat the subject than when compared to the administration of said laquinimod in the absence of said fingolimod or the administration of said fingolimod in the absence of said laquinimod.

This invention also provides a pharmaceutical composition in unit dosage form, useful in treating a subject afflicted with a neurodegenerative disease, which comprises: a) an amount of laquinimod; b) an amount of fingolimod, wherein the respective amounts of said laquinimod and said fingolimod in said composition are effective, upon concomitant administration to said subject of one or more of said unit dosage forms of said composition, to treat the subject. In an embodiment, the respective amounts of said laquinimod and said fingolimod in said unit dose when taken together is more effective to treat the subject than when compared to the administration of said laquinimod in the absence of said fingolimod or the administration of said fingolimod in the absence of said laquinimod.

For the foregoing embodiments, each embodiment disclosed herein is contemplated as being applicable to each of the other disclosed embodiment. For example, the elements recited in the method embodiments can be used in the use, composition and package embodiments described herein and vice versa.

### Fingolimod

Fingolimod mixtures, compositions, the process for the manufacture thereof, the use thereof for treatment of various conditions, and the corresponding dosages and regimens are described in, e.g., U.S. Patent Application Publication Nos. 2012-0184617, 2009-0176744, 2009-0082347, and 2011-0152380, U.S. Pat. No. 5,719,176, and Pelletier and Hafler (2012) “Fingolimod for Multiple Sclerosis” New England Journal of Medicine, 366(4):339-347, each of which is hereby incorporated by reference in its entireties into this application.

### Laquinimod

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in its entireties into this application.

Use of laquinimod for treatment of various conditions, and the corresponding dosages and regimens, are described in U.S. Pat. No. 6,077,851 (multiple sclerosis, insulin-dependent diabetes mellitus, systemic lupus erythematosus, rheumatoid arthritis, inflammatory bowel disease, psoriasis, inflammatory respiratory disorder, atherosclerosis, stroke, and Alzheimer's disease), U.S. Application Publication No. 2011-0027219 (Crohn's disease), U.S. Application Publication No. 2010-0322900 (relapsing-remitting multiple sclerosis), U.S. Application Publication No. 2011-0034508 (brain-derived neurotrophic factor (BDNF)-related diseases), U.S. Application Publication No. 2011-0218179 (active lupus nephritis), U.S. Application Publication No. 2011-0218203 (rheumatoid arthritis), U.S. Application Publication No. 2011-0217295 (active lupus arthritis), and U.S. Application Publication No. 2012-0142730 (reducing fatigue, improving quality of life, and providing neuroprotection in MS patients), each of which is hereby incorporated by reference in its entireties into this application.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit can be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248.

General techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol. 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a subject, e.g., human patient, afflicted with a neurodegenerative disease using laquinimod with fingolimod which provides a more efficacious treatment than each agent alone. The use of laquinimod for multiple sclerosis had been previously suggested in, e.g., U.S. Pat. No. 6,077,851. The use of laquinimod for certain neurodegenerative diseases, i.e., PD, HD, ALS and AD, had been previously suggested in, e.g., U.S. Patent Application Publication No. 2011-0034508. However, the inventors have surprisingly found that the combination of laquinimod and fingolimod is particularly effective as compared to each agent alone.

### Terms

As used herein, and unless stated otherwise, each of the following terms shall have the definition set forth below.

As used herein, “laquinimod” means laquinimod acid or a pharmaceutically acceptable salt thereof.

As used herein, “fingolimod” or “FTY 720” means fingolimod acid or a pharmaceutically acceptable salt thereof.

As used herein, an “amount” or “dose” of laquinimod or fingolimod as measured in milligrams refers to the milligrams of laquinimod or fingolimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion. Similarly, when in the form of a salt, e.g. fingolimod hydrochloride, the weight of the salt form necessary to provide a dose of 0.5 mg fingolimod would be greater than 0.5 mg (e.g., 0.56 mg) due to the presence of the additional salt ion.

As used herein, a “unit dose”, “unit doses” and “unit dosage form(s)” mean a single drug administration entity/entities.

As used herein, “about” in the context of a numerical value or range means±10% of the numerical value or range recited or claimed.

As used herein, a composition that is “free” of a chemical entity means that the composition contains, if at all, an amount of the chemical entity which cannot be avoided although the chemical entity is not part of the formulation and was not affirmatively added during any part of the manufacturing process. For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound selected from the group consisting of tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein also refers to Flavonoids such as those selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound selected from the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.”

As used herein, “chelating agent” refers to a compound selected from the group consisting of penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), desferal from Novartis (previously Ciba-Giegy), and apoferritin.

As used herein, a pharmaceutical composition is “stable” when the composition preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the laquinimod and the fingolimod. In this case, the combination may be the admixture or separate containers of the laquinimod and the fingolimod that are combined just prior to administration. Contemporaneous administration refers to the separate administration of the laquinimod and the fingolimod at the same time, or at times sufficiently close together that a synergistic activity relative to the activity of either the laquinimod or the fingolimod alone is observed.

As used herein, “concomitant administration” or administering “concomitantly” means the administration of two agents given in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding laquinimod therapy to a patient already receiving fingolimod therapy or adding fingolimod therapy to a patient already receiving laquinimod therapy.

As used herein, “effective” when referring to an amount of laquinimod and/or fingolimod refers to the quantity of laquinimod and/or fingolimod that is sufficient to yield a desired therapeutic response without undue adverse side effects (such as toxicity, irritation, or allergic response) commensurate with a reasonable benefit/risk ratio when used in the manner of this invention.

“Administering to the subject” or “administering to the (human) patient” means the giving, dispensing, or application of medicines, drugs, or remedies to a subject/patient to relieve, cure, or reduce the symptoms associated with a condition, e.g., a pathological condition.

“Treating” as used herein encompasses, e.g., inducing inhibition, regression, or stasis of a disease or disorder, e.g., AD, ALS, HD or PD, or alleviating, lessening, suppressing, inhibiting, reducing the severity of, eliminating or substantially eliminating, or ameliorating a symptom of the disease or disorder.

“Inhibition” of disease progression or disease complication in a subject means preventing or reducing the disease progression and/or disease complication in the subject.

A “symptom” associated with AD, ALS, HD or PD includes any clinical or laboratory manifestation associated with AD, ALS, HD or PD and is not limited to what the subject can feel or observe.

As used herein, “a subject afflicted with” a neurodegenerative disease, e.g., AD, ALS, HD or PD, means a subject who has been clinically diagnosed to have said neurodegenerative disease.

“Neurodegenerative disease” is defined herein as a disorder in which progressive loss of neurons occurs either in the peripheral nervous system (PNS) or in the central nervous system (CNS). Non-limiting examples of neurodegenerative diseases include chronic neurodegenerative diseases such as familial and sporadic Parkinson's disease, Huntington's disease, familial and sporadic Amyotrophic lateral sclerosis (FALS and ALS, respectively), familial and sporadic Alzheimer's disease. The foregoing examples are not meant to be comprehensive but serve merely as an illustration of the term.

In an embodiment of the present invention “neurodegenerative disease” includes a form of multiple sclerosis. In another embodiment, “neurodegenerative disease” excludes any form of multiple sclerosis.

As used herein, a subject at “baseline” is as subject prior to administration of laquinimod or fingolimod.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Mechanism of Action Studies of Laquinimod

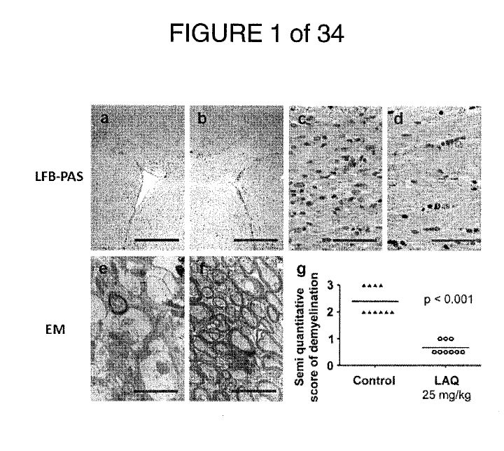

In one study, the effect of laquinimod on demyelination in the cuprizone model (non-T cell model of demyelination) was that the laquinimod treatment results in significantly less demyelination, as presented in FIG. 1.

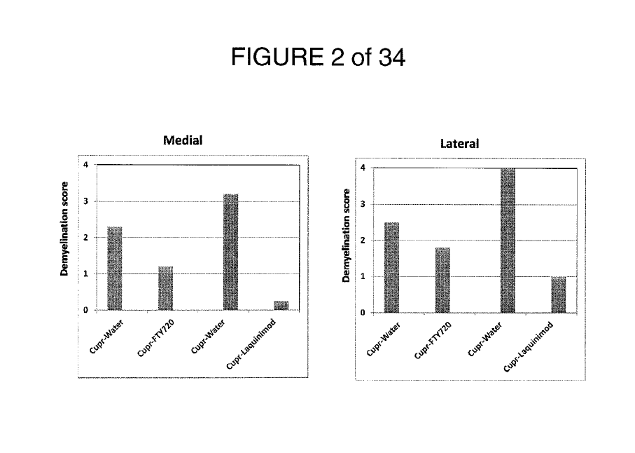

In another study, pooled data were published on demyelination score (Brück, 2012). FIG. 2 shows demyelination in lateral and medial corpus callosum separately.

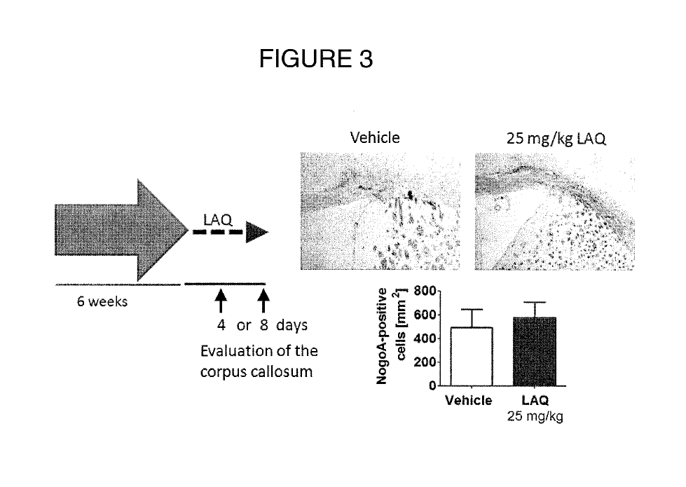

In another study, the effect of laquinimod on re-myelination in the cuprizone model (non-T cell model of demyelination) was that there was no effect by laquinimod on re-myelination after cuprizone withdrawal, as presented in FIG. 3.

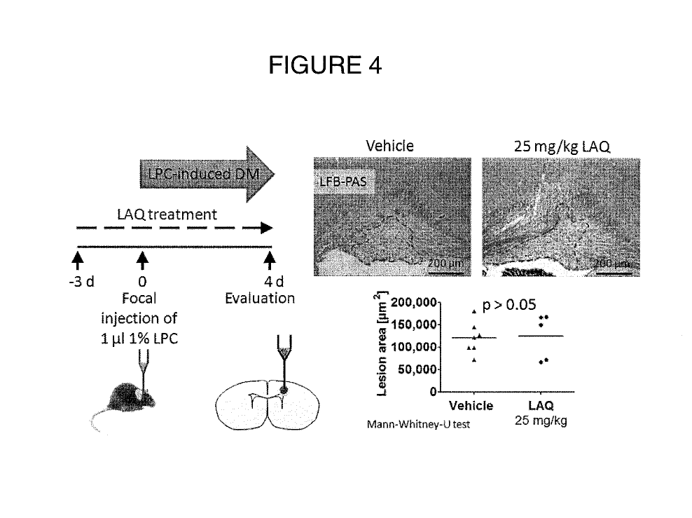

In another study, the effect of laquinimod on lysolecithin-induced demyelination was that there was no effect by laquinimod on demyelination in the lysolecithin model, as presented in FIG. 4.

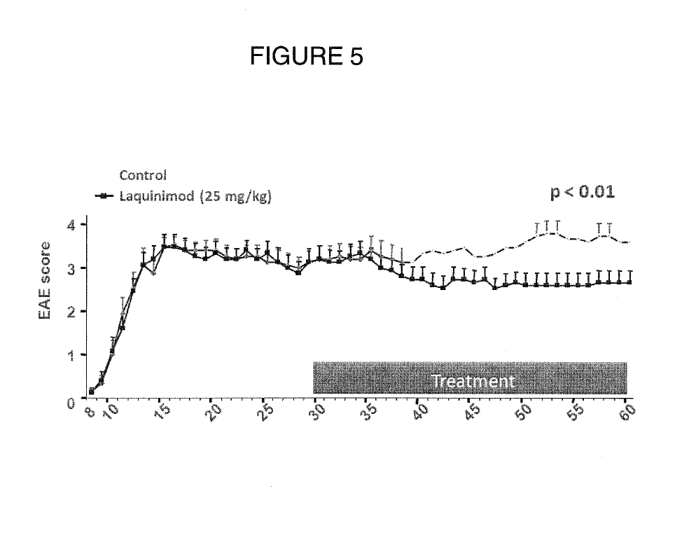

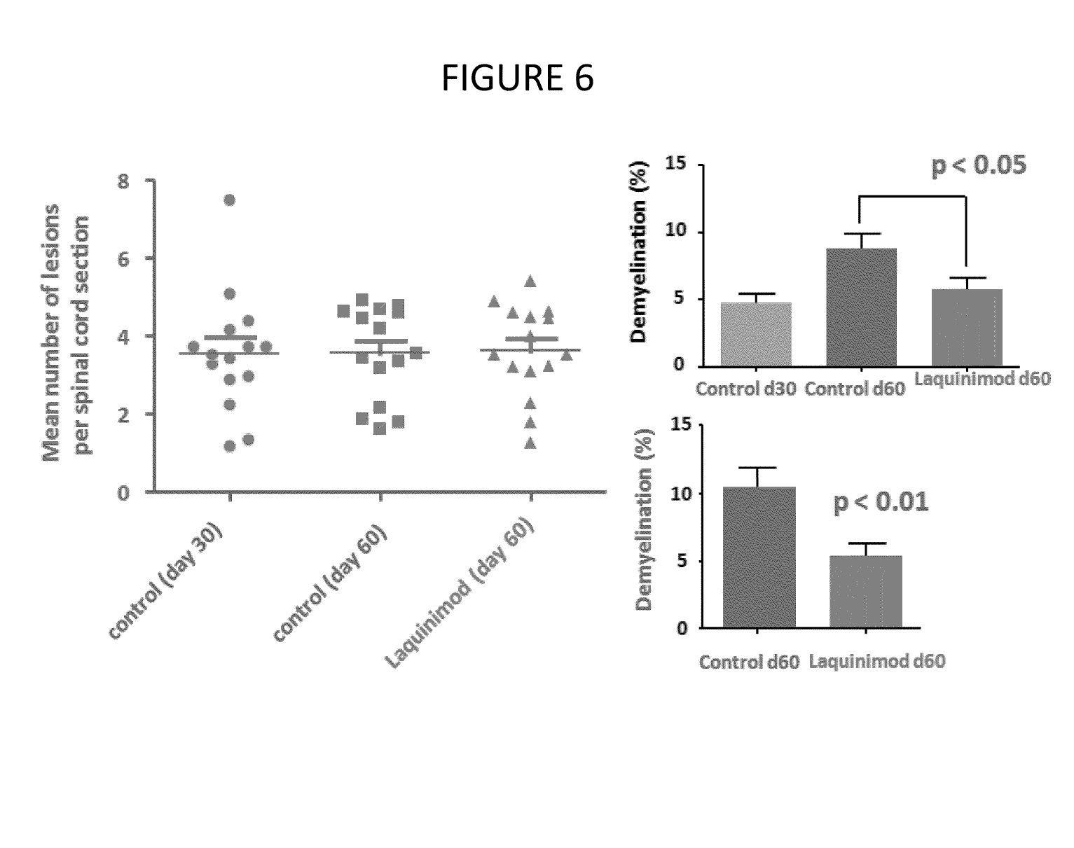

In another study, the effect of laquinimod on established EAE was that the treatment ameliorates clinical disease in EAE and inhibits further expansion of pre-existing lesions, as presented in FIGS. 5 and 6.

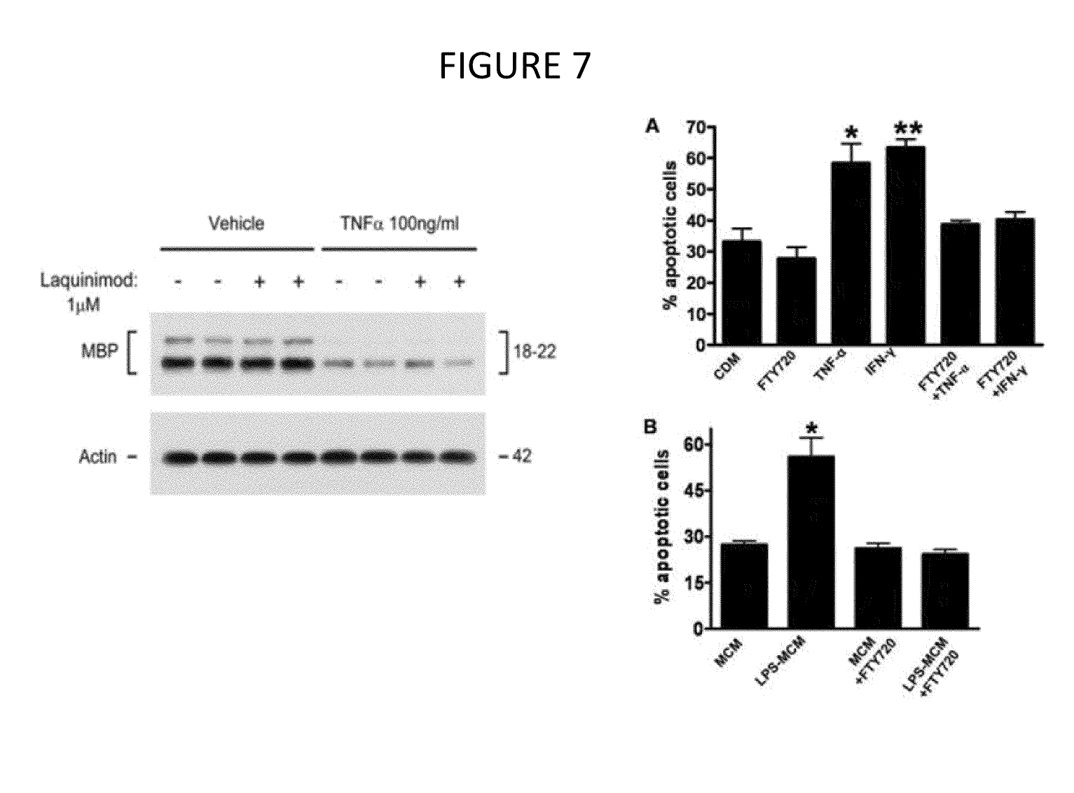

In another study, the effect of laquinimod on oligodendrocyte survival was that the treatment does not protect oligodendrocytes from inflammatory insults, as presented in FIG. 7.

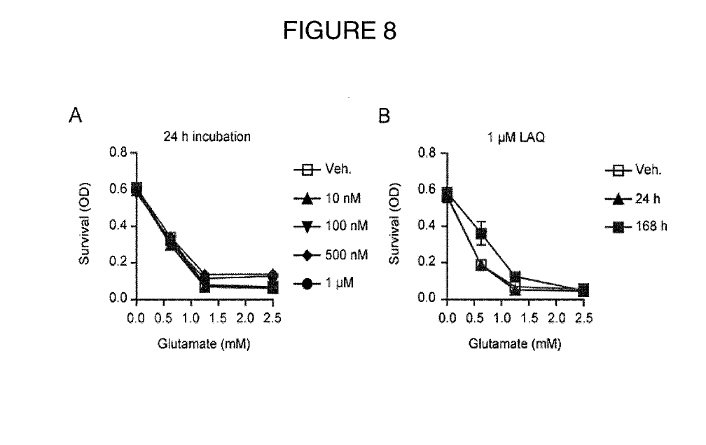

In another study, the effect of laquinimod on oxidative glutamate toxicity of H2TT (primary neuronal culture) cells was that prolonged incubation with laquinimod protects against oxidative glutamate toxicity, as presented in FIG. 8.

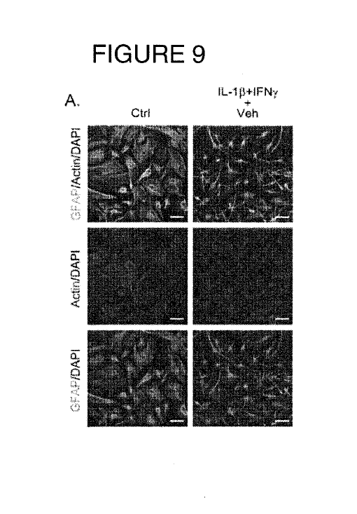

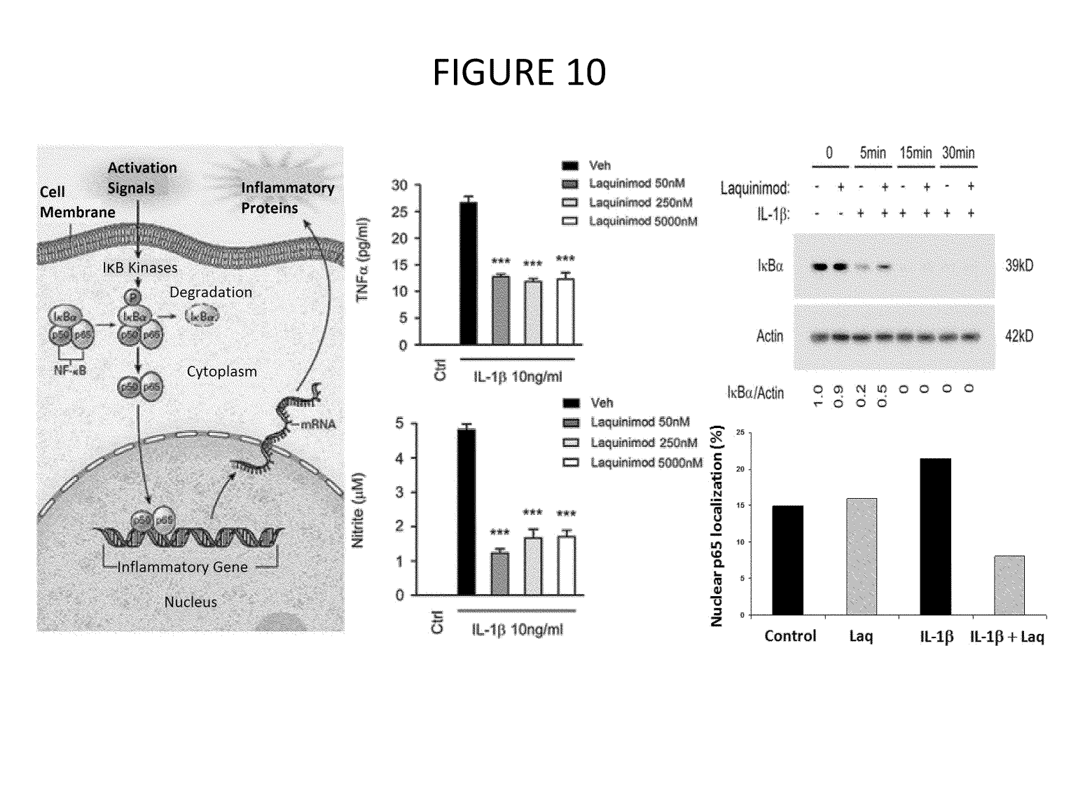

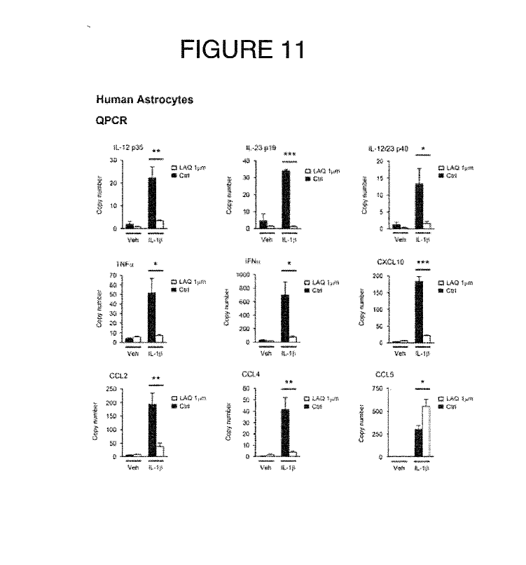

The effect of laquinimod on human astrocyte activation, as investigated in another study, is detailed in FIGS. 9 and 10. Laquinimod interferes with astrocyte activation via the NF-κB pathway. In unstimulated cells, the NF-κB dimers are sequestered in the cytoplasm by a family of inhibitors, called IκBs (Inhibitor of κB). The IκB proteins mask the nuclear localization signals (NLS) of NF-κB proteins and keep them sequestered in an inactive state in the cytoplasm. Activation of the NF-κB is initiated by the signal-induced degradation of IκB proteins. This occurs primarily via activation of a kinase called the IκB kinase (IKK). When activated by signals, usually coming from the outside of the cell, the IκB kinase phosphorylates two serine residues located in an IκB regulatory domain. When phosphorylated, the IκB inhibitor molecules are modified by a process called ubiquitination, which than leads them to be degraded by a cell structure called the proteasome. With the degradation of IκB, the NF-κB complex is then freed to enter the nucleus where it can ‘turn on’ the expression of specific genes that have DNA-binding sites for NF-κB nearby. The activation of these genes by NF-κB then leads to the given physiological response, for example, an inflammatory or immune response. In another study, it was shown that laquinimod down regulates pro-inflammatory cytokine secretion from human astrocytes in vitro, as presented in FIG. 11.

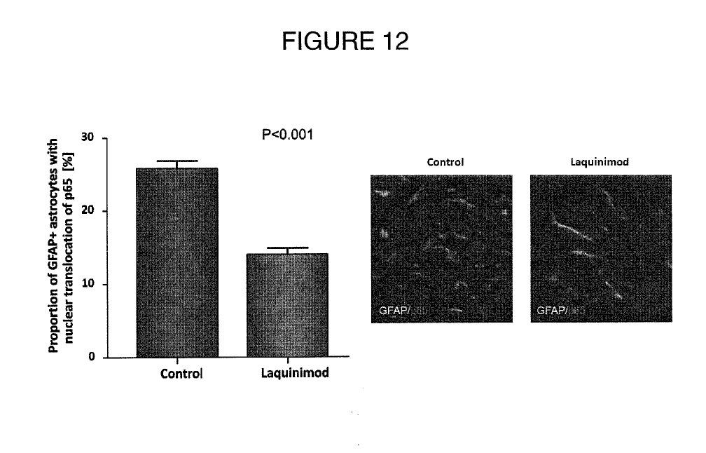

In another study, the effect of laquinimod on p65 translocation into the astrocyte nucleus in vivo was that the treatment results in significantly reduced astrocyte activation via interference with the NF-κB pathway, as presented in FIG. 12.

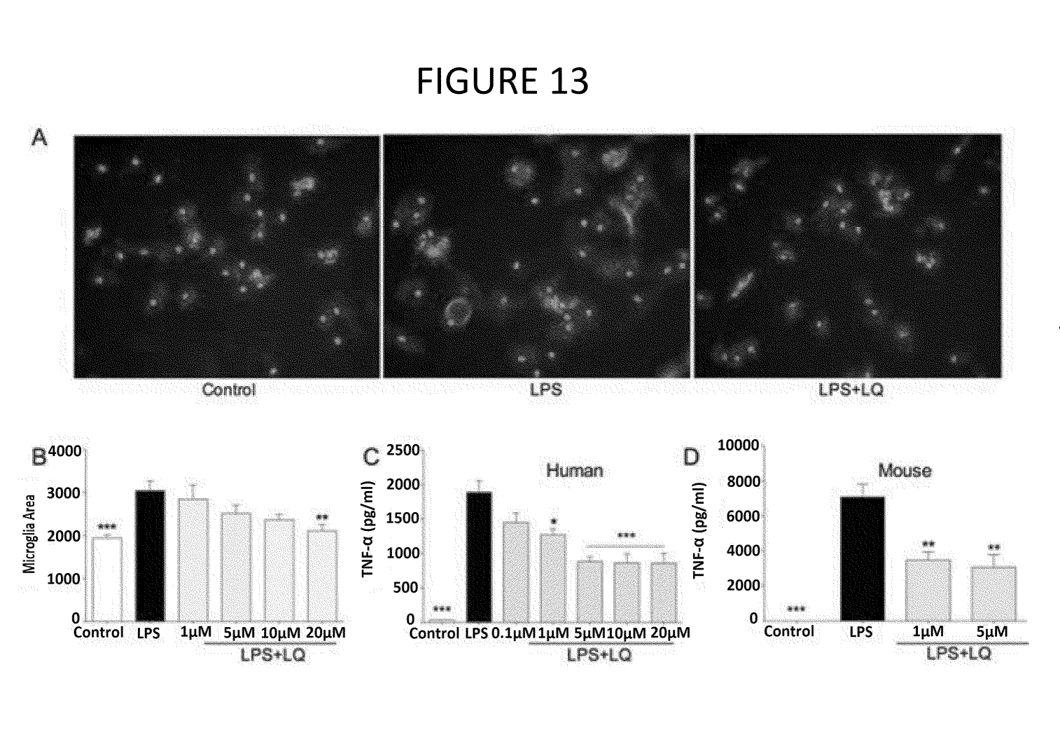

In another study, laquinimod reduced microglial activation in culture. The size of CD14 stained human microglia was increased with LPS activation. This effect was reduced by laquinimod (A-B), as presented in FIG. 13. Also, in FIG. 13, human (C) or mouse (D) microglia elevated TNF-α secretion upon LPS activation, which was attenuated by laquinimod.

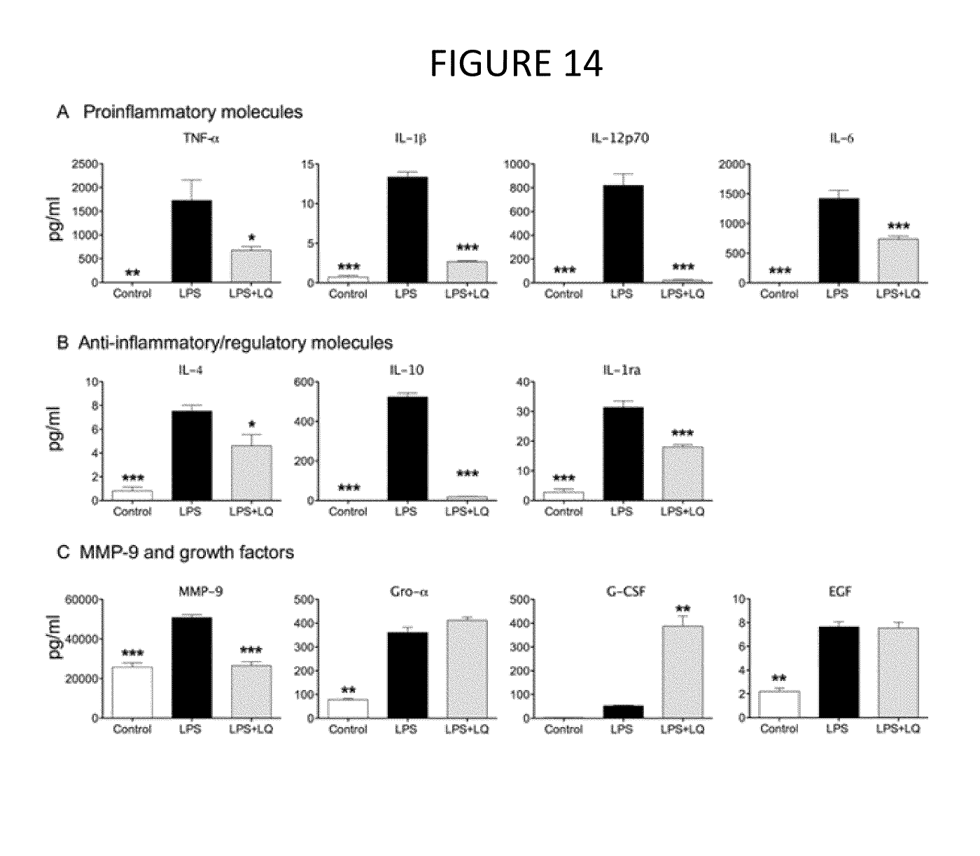

Laquinimod inhibited microglial production of pro-inflammatory cytokine in human microglia, as presented in FIG. 14.

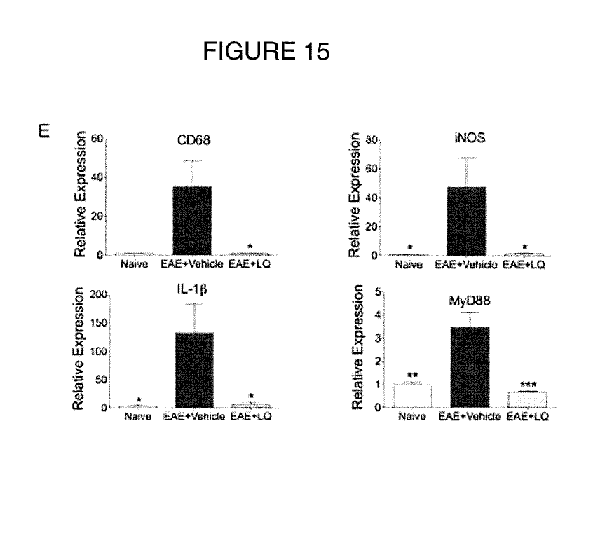

Laquinimod inhibited microglial activation in EAE in mice. Transcripts encoding markers of activation of microglia/macrophages were increased in the spinal cord of EAE-afflicted mice and decreased in laquinimod-treated animals, as presented in FIG. 15.

A number of diseases have been suggested in the art to be linked to astrocyte and/or microglia malfunction. These diseases include but are not limited to Alzheimer's disease, Amyotrophic lateral sclerosis, Huntington's disease, Parkinson's disease, Alexander disease, certain types cerebellar ataxia including spinocerecellar ataxia (SCA), Batten disease, Creutzfeldt-Jakob disease, Charcot-Marie-Tooth disease (CMT), HIV-associated dementia, multiple system atrophy (MSA) and prion-related disease (Amor et al., 2010; Barbierato, 2012; Barreto et al., 2011; Carson et al., 2006; Cerbai, 2012; Giuliano, 2011; Liu and Hong, 2003; Lobsiger, 2007; Maragakis and Rothstein, 2006; Mattson and Camandola, 2001 and Taboada et al., 2011). The treatment of these diseases according to the methods and uses as disclosed herein are within the scope of the present invention.

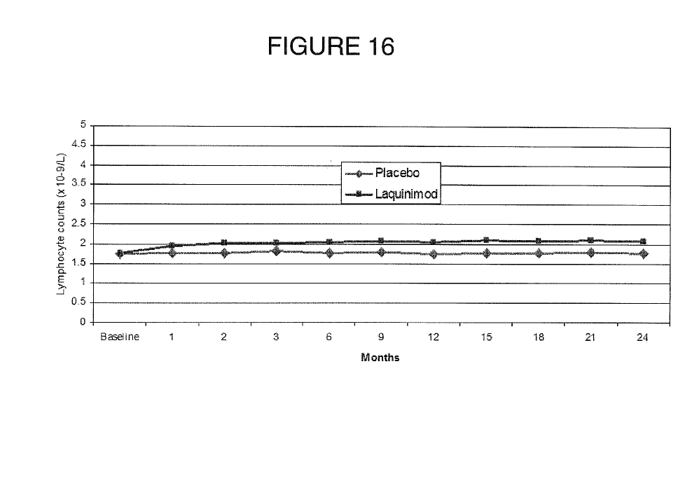

In another study, lymphocyte counts remained stable over time with laquinimod, with no clinically significant difference in mean group levels of lymphocyte counts in the laquinimod 0.6 mg group as compared with a placebo or with the baseline at all visits, as presented in FIG. 16.

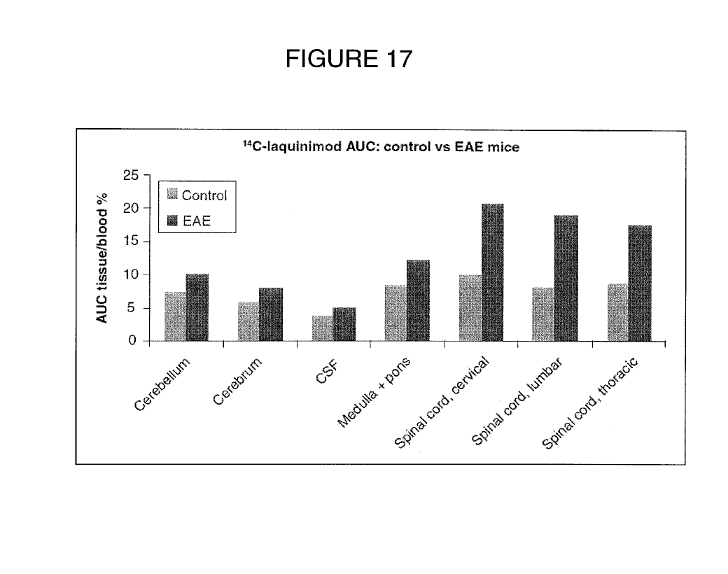

In another study, laquinimod, as a small molecule, penetrated both intact and disrupted Blood Brain Barrier (BBB). In view of FIG. 17, CNS tissue level of laquinimod was 7-8% of the blood concentration in healthy mice and 13% in EAE mice when the BBB was disrupted. Further, 90% of the drug in cerebrospinal fluid (CSF) was active, or free, due to low protein binding and expected also in brain interstitial fluids. Thus, laquinimod targeted the entire brain and not only the lesions.

### Example 2

### Mechanism of Action Studies of Fingolimod

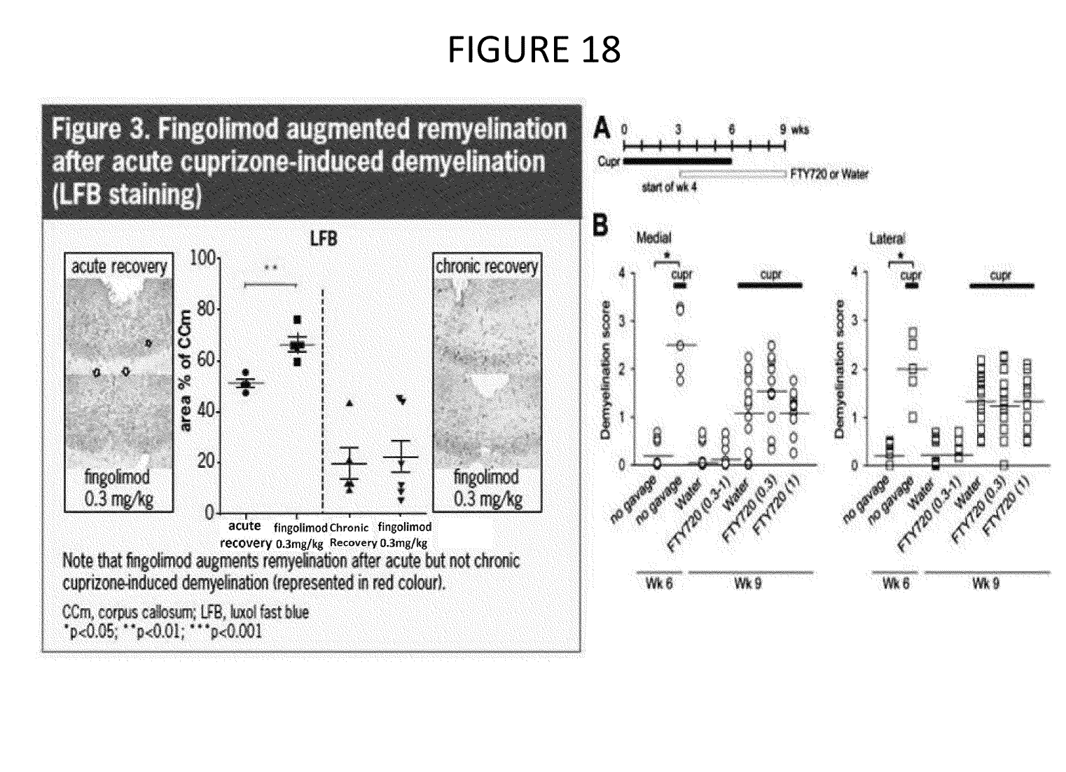

In several studies, the effect of fingolimod (FTY 720) on re-myelination in the cuprizone model was not conclusive, as presented in FIG. 18 (Slovic, 2012; Kim, 2011).

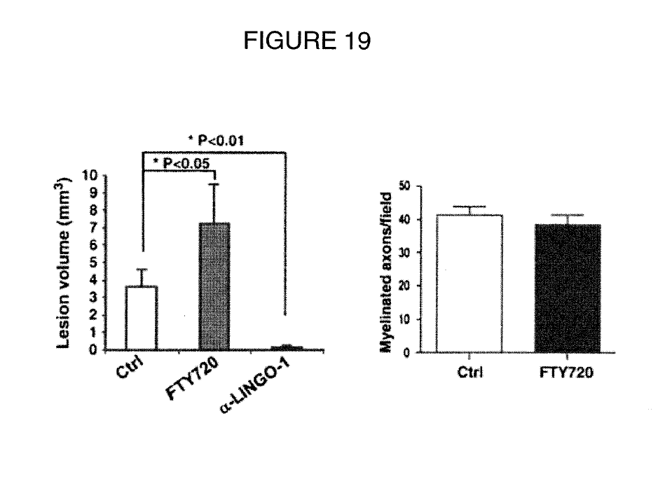

In another study, the effect of FTY 720 on re-myelination in the lysolecithin-induced demyelination model was a lack of any effect by FTY 720 on re-myelination in the lysolecithin model, as presented in FIG. 19 (Hu, 2011).

In another study, the effect of FTY 720 on oligodendrocyte survival was that the treatment did protect oligodendrocytes from inflammatory insults, as presented in FIG. 7 (Rochelle, 2007).

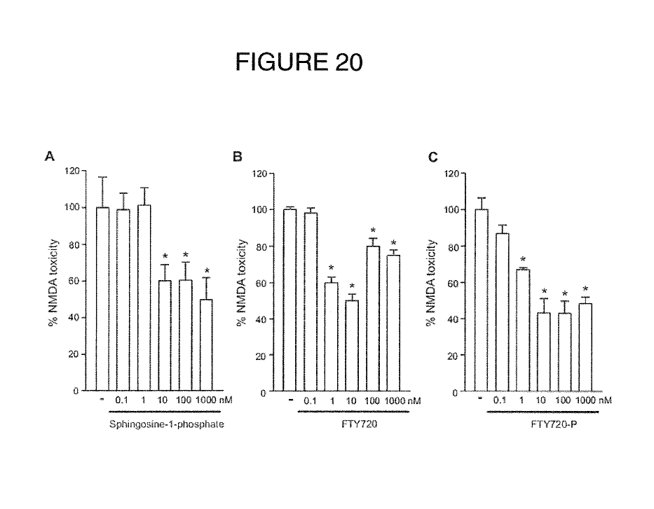

In another study, the effect of S1P, FTY 720, or FTY 720-P in the pretreatment of mouse-cultured cortical cells was that the inclusion of S1P, FTY 720, or FTY 720-P protected neurons against NMDA toxicity, as presented in FIG. 20 (Di Menna, 2013).

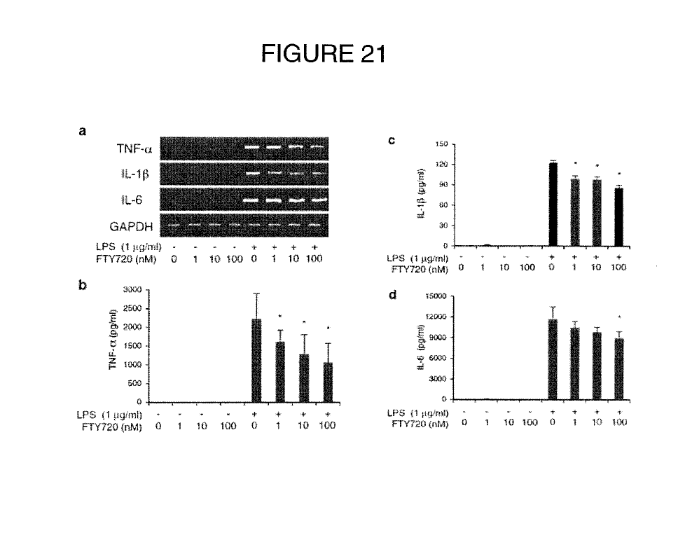

In another study, FTY 720 inhibited microglial production of pro-inflammatory cytokine in mouse primary microglia, as presented in FIG. 21.

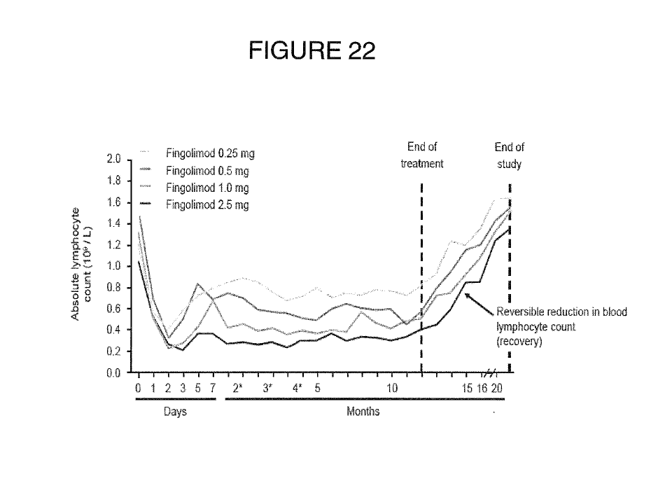

In another study, reduction in peripheral lymphocyte counts by fingolimod was found to be SIP receptor-mediated, and therefore, lymphocyte count reduction by fingolimod is dose dependent, as presented in FIG. 22.

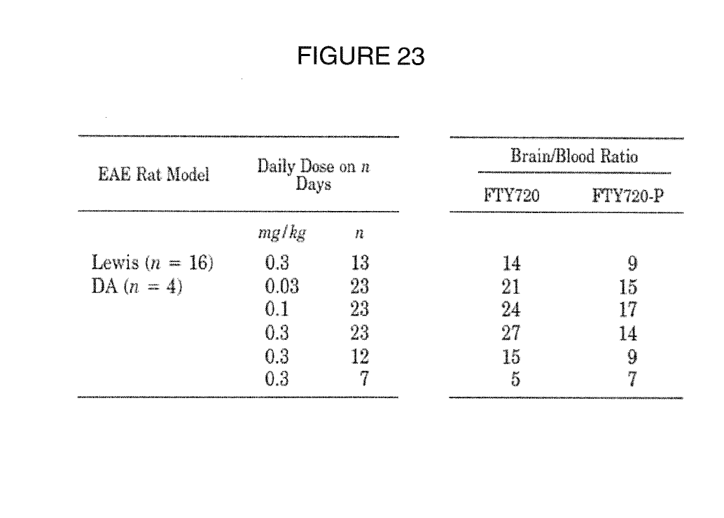

In another study, there was a high brain/plasma ratio of fingolimod in Dark Agouti (DA) experimental autoimmune encephalomyelitis (EAE) induced rats, where the brain/blood ratio of fingolimod when administered at doses 0.03-0.3 mg/kg was about 20, as presented in FIG. 23 (Foster, 2007). There is no data available, however, on CNS exposure of fingolimod in animals with intact CNS.

### Example 3

### Comparison of Mechanism of Action of Laquinimod and Fingolimod

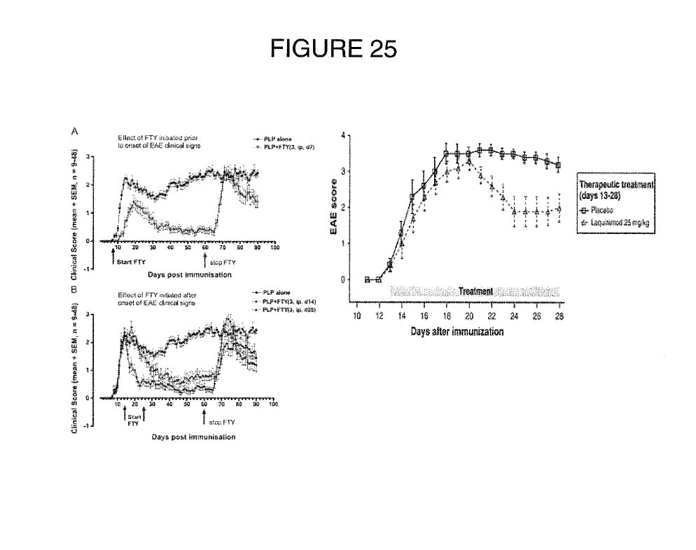

Examples 1 and 2 demonstrate that laquinimod and fingolimod have different mechanism of action (MoA) in chronic EAE as presented in FIG. 25 (Webb, 2004; Wegner, 2010). In addition, laquinimod and fingolimod exhibit partial effect on many neuroprotective parameters.

Fingolimod has major peripheral anti-inflammatory, and consequently, neuroprotective effects in relapsing-remitting multiple sclerosis (RAMS). In addition, fingolimod has some direct CNS effects, which are not only the consequence of peripheral immune effects. In contrast, laquinimod has major direct CNS effects with relatively lower peripheral anti-inflammatory effects in RRMS.

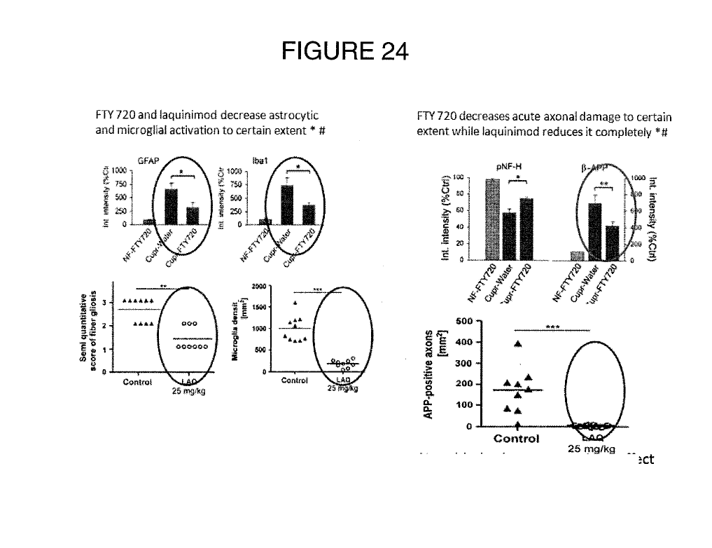

Each of FTY 720 and laquinimod decrease demyelination, astrocytic and microglial activation by a certain amount (partial response), as presented in FIG. 24 (Kim, 2011). In contrast, FTY 720 decreases acute axonal damage by a certain amount, while laquinimod reduces it completely (Brück, 2012).

### Example 4

### Co-Administration of Laquinimod and Fingolimod

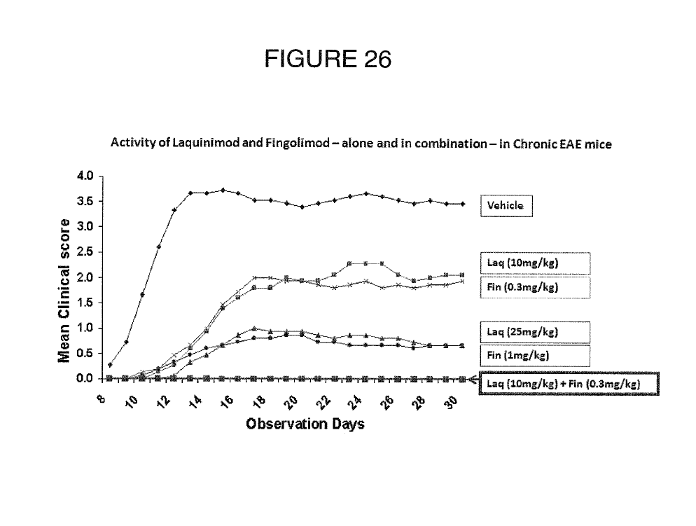

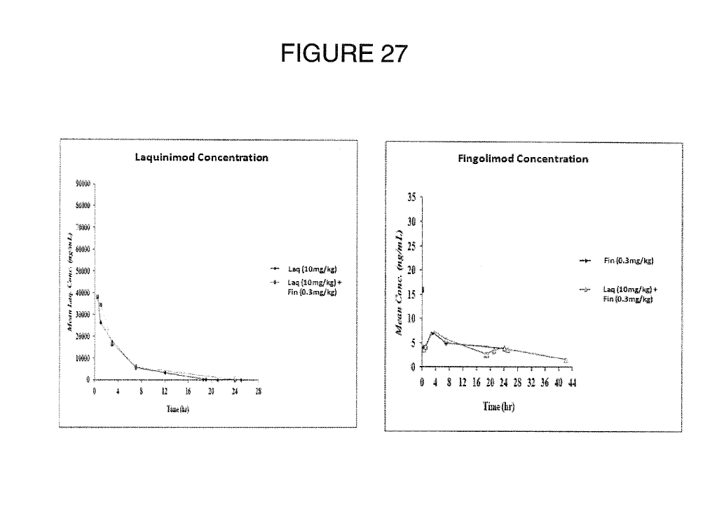

In one study the co-administration of laquinimod and fingolimod, remarkably reduced the clinical score in EAE-induced animal model of inflammation, as presented in FIG. 26. Further, according to FIG. 27, there were no drug-drug interactions, and the pharmacokinetic (PK) attributes (e.g., levels, half-life and AUC) of each drug were not affected by concomitant administration, which means there was no change in metabolic rates.

### Example 5

### Animal Models of Neurodegenerative Diseases

### Example 5.1

### Assessment of Efficacy of Laquinimod and Fingolimod in an Animal Model of AD

Transgenic mouse models of Alzheimer disease have been invaluable in unraveling the mechanisms of disease progression and for testing potential therapeutic interventions. Since the cause of sporadic AD is unknown, transgenic models of AD are primarily based on mutations found only in patients with familial AD. These mutations produce pathological and cognitive changes that resemble sporadic AD, and thus these transgenic mice are still extremely useful for studying this more common form of AD. Transgenic models of AD, such as the finding from 3×Tg-AD mice and other models have demonstrated that tau pathology is facilitated by amyloid-β (Avila et al., 2011).

Senile plaques and neurofibrillary tangles (NFTs) are major pathological proteinaceous anomalies that occur in the brains of AD patients. Motivated by the amyloid hypothesis, animal models exhibiting Aβ deposition have been produced by crossbreeding mice over-expressing human mutant amyloid precursor protein (hAPP) with mice over-expressing mutant PS-1, the latter of which accelerates Aβ deposition in the brain. Most mouse models exhibiting Aβ deposition show memory deficits associated with synaptic plasticity impairments and synapse loss (Avila et al., 2011).

Reelin is an extracellular protein crucial for brain development. To study Reelin functions in the adult forebrain a transgenic mouse model was generated that over-express Reelin under the control of the CaMKIIα promoter (pCaMKII-Reelin-OE; Tg1/Tg2)1. Studies on Tg1/Tg2 mice indicate that Reelin regulates adult neurogenesis and migration, as well as the structural and functional properties of synapses. These observations suggest that Reelin controls developmental processes that remain active in the adult brain (Avila et al., 2011).

An amount of laquinimod, an amount of fingolimod or an amount of both laquinimod and fingolimod is administered to transgenic mice models of Alzheimer's disease (e.g., an amyloid/PS-1 transgenic mice model or transgenic mice over-expressing GSK-3β or Reelin). The combination of laquinimod and fingolimod provides at least an additive effect or more than an additive effect in treating the animal model of AD.

### Example 5.2

### Assessment of Efficacy of Laquinimod and Fingolimod in an Animal Model of ALS

There are growing numbers of reports on ALS animal models. Most of them are rodent transgenic models over-expressing ALS-associated mutant genes, either constitutively or conditionally (Avila et al., 2011).

An amount of laquinimod, an amount of fingolimod or an amount of both laquinimod and fingolimod is administered to transgenic mice models of ALS (e.g., SOD1 microinjected rat). The combination of laquinimod and fingolimod provides at least an additive effect or more than an additive effect in treating the animal model of ALS.

### Example 5.3

### Assessment of Efficacy of Laquinimod and Fingolimod in an Animal Model of HD

Earlier studies of HD most often used toxin-induced models to study mitochondrial impairment and excitotoxicity-induced cell death, which are both mechanisms of degeneration seen in the HD brain. These models, based on 3-nitropropionic acid and quinolinic acid, respectively, are still often used in HD studies. The discovery of the huntingtin mutation led to the creation of newer models that incorporate a similar genetic defect. These models, which include transgenic and knock-in rodents, are more representative of the HD progression and pathology. An even more recent model that uses a viral vector to encode the gene mutation in specific areas of the brain may be useful in nonhuman primates, as it is difficult to produce genetic models in these species (Ramaswamy, 2007).

An amount of laquinimod, an amount of fingolimod or an amount of both laquinimod and fingolimod is administered to an excitotoxic (e.g., quinolinic acid) model of HD, transgenic mice models of HD or a Knock-In model created by insertion of CAG repeats. The combination of laquinimod and fingolimod provides at least an additive effect or more than an additive effect in treating the animal model of HD.

### Example 5.4

### Assessment of Efficacy of Laquinimod and Fingolimod in an Animal Model of PD

Multiples genetic approaches exist to model the rare familial autosomal dominant (e.g. transgenic and targeted over-expression of the mutant gene of interest; α-synuclein or LRRK2); and recessive cases of PD (targeted deletion of the relevant gene; e.g. parkin, DJ-1, etc.). Alternatively, toxins causing broad or dopamine neuron-specific mitochondrial dysfunction have been employed to model the complex I deficiency reported in sporadic cases of PD; or those that impair proteasomal-based protein degradation effectively model the formation of neuronal Lewy bodies (Avila et al., 2011). An amount of laquinimod, an amount of fingolimod or an amount of both laquinimod and fingolimod is administered to transgenic mice models of PD (e.g., α-synuclein transgenic mice) or toxic models (6-hydroxydopamine or 6-OHDA) of lesion rats. The combination of laquinimod and fingolimod provides at least an additive effect or more than an additive effect in treating the animal model of PD.

### Example 6

### Assessment of Efficacy of Laquinimod and Fingolimod Add-on and Combination Therapy in Neurodegenerative Diseases

Combined dosing of laquinimod and fingolimod, each with an independent Mechanism of Action (MoA), provides at least an additive effect or more than an additive effect, and allows for dose reduction of each drug used.

The Examples above demonstrate that laquinimod and fingolimod have different MoAs and exhibit partial effect on many neuroprotective parameters, e.g., microglial and astrocytic activation. The combined therapy using laquinimod and fingolimod demonstrates at least an additive effect or more than an additive effect.

Combined dosing also provides high brain/blood exposure (of fingolimod) and high free active fraction (of laquinimod) in the CNS, achieving anti-inflammatory activity in the CNS, reducing lesion foci number and extent of their pathology (by fingolimod), slowing neurodegeneration in the entire brain, and reducing brain tissue loss (by laquinimod).

### Example 6.1

### Assessment of Efficacy of Laquinimod as Add-on Therapy to Fingolimod and Fingolimod as Add-on Therapy to Laquinimod in AD Patients

The add-on therapy provides a synergistic effect, and allow for lower doses with reduced side effects.

Periodic administration of laquinimod (p.o. 0.1, 0.15, 0.2, 0.25, 0.3 or 0.6 mg/day) as an add-on therapy for a human patient afflicted with AD who is already receiving fingolimod (p.o. 0.05, 0.1, 0.15, 0.2, 0.25, 0.3, 0.4, or 0.5 mg/day) provides a clinically meaningful advantage and is more effective (provides at least an additive effect or more than an additive effect) in treating the patient than when fingolimod is administered alone (at the same dose).

Periodic administration fingolimod (p.o. 0.05, 0.1, 0.15, 0.2, 0.25, 0.3, 0.4, or 0.5 mg/day) as an add-on therapy for a human patient afflicted with AD who is already receiving of laquinimod (p.o. 0.1, 0.15, 0.2, 0.25, 0.3 or 0.6 mg/day) provides a clinically meaningful advantage and is more effective (provides at least an additive effect or more than an additive effect) in treating the patient than when laquinimod is administered alone (at the same dose).

The add-on therapies also provides efficacy (provides at least an additive effect or more than an additive effect) in treating the patient without undue adverse side effects or affecting the safety of the treatment. As compared to when each agent is administered alone:
1. The add-on therapy is more effective (provides an additive effect or more than an additive effect) in reducing the decrease in brain volume (determined by the percent brain volume change (PBVC)), in AD.
2. The add-on therapy is more effective (provides an additive effect or more than an additive effect) in maintaining, preventing or slowing the deterioration of, or improving memory, in AD patients.
3. The add-on therapy is more effective (provides an additive effect or more than an additive effect) in maintaining or improving cognitive function in AD patients.
4. The add-on therapy is more effective (provides an additive effect or more than an additive effect) in reversing, preventing or slowing cognitive impairment in AD patients.
5. The add-on therapy is more effective (provides an additive effect or more than an additive effect) in reversing, preventing or slowing functional impairment in AD patients.
6. The add-on therapy is more effective (provides an additive effect or more than an additive effect) in delaying time to onset of dementia in AD patients.

### Example 6.2

### Assessment of Efficacy of Laquinimod in Combination with Fingolimod in AD Patients

The combination therapy provides a synergistic effect, and allow for lower doses with reduced side effects.

Periodic administration of laquinimod (p.o. 0.1, 0.15, 0.2, 0.25, 0.3 or 0.6 mg/day) in combination with fingolimod (p.o. 0.05, 0.1, 0.15, 0.2, 0.25, 0.3, 0.4, or 0.5 mg/day) to a human patient afflicted with AD provides increased efficacy (provides at least an additive effect or more than an additive effect) in treating the patient than when laquinimod is administered alone or when fingolimod is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when laquinimod or fingolimod is administered alone (at the same dose) in the following manner:
1. The combination therapy is more effective (provides an additive effect or more than an additive effect) in reducing the decrease in brain volume (determined by the percent brain volume change (PBVC)), in AD.
2. The combination therapy is more effective (provides an additive effect or more than an additive effect) in maintaining, preventing or slowing the deterioration of, or improving memory, in AD patients.
3. The combination therapy is more effective (provides an additive effect or more than an additive effect) in maintaining or improving cognitive function in AD patients.
4. The combination therapy is more effective (provides an additive effect or more than an additive effect) in reversing, preventing or slowing cognitive impairment in AD patients.
5. The combination therapy is more effective (provides an additive effect or more than an additive effect) in reversing, preventing or slowing functional impairment in AD patients.
6. The combination therapy is more effective (provides an additive effect or more than an additive effect) in delaying time to onset of dementia in AD patients.

### Example 6.3

### Assessment of Efficacy of Laquinimod as Add-on Therapy to Fingolimod and Fingolimod as Add-on Therapy to Laquinimod in ALS Patients

The add-on therapy provides a synergistic effect, and allow for lower doses with reduced side effects.

Periodic administration of laquinimod (p.o. 0.1, 0.15, 0.2, 0.25, 0.3 or 0.6 mg/day) as an add-on therapy for a human patient afflicted with ALS who is already receiving fingolimod (p.o. 0.05, 0.1, 0.15, 0.2, 0.25, 0.3, 0.4, or 0.5 mg/day) provides a clinically meaningful advantage and is more effective (provides at least an additive effect or more than an additive effect) in treating the patient than when fingolimod is administered alone (at the same dose).

Periodic administration fingolimod (p.o. 0.05, 0.1, 0.15, 0.2, 0.25, 0.3, 0.4, or 0.5 mg/day) as an add-on therapy for a human patient afflicted with ALS who is already receiving of laquinimod (p.o. 0.1, 0.15, 0.2, 0.25, 0.3 or 0.6 mg/day) provides a clinically meaningful advantage and is more effective (provides at least an additive effect or more than an additive effect) in treating the patient than when laquinimod is administered alone (at the same dose).

The add-on therapies also provides efficacy (provides at least an additive effect or more than an additive effect) in treating the patient without undue adverse side effects or affecting the safety of the treatment. As compared to when each agent is administered alone:
1. The add-on therapy is more effective (provides an additive effect or more than an additive effect) in prolonging survival of ALS patients.
2. The add-on therapy is more effective (provides an additive effect or more than an additive effect) in maintaining or improving the ALS Functional Rating Scale-Revised (ALSFRS-R) total score in the subject.
3. The add-on therapy is more effective (provides an additive effect or more than an additive effect) in reversing, preventing or slowing motor neuron damage in the subject.
4. The add-on therapy is more effective (provides an additive effect or more than an additive effect) in reversing, preventing or slowing functional impairment the subject.
5. The add-on therapy is more effective (provides an additive effect or more than an additive effect) in reversing, preventing or slowing motor function impairment in HD patients.
6. The add-on therapy is more effective (provides an additive effect or more than an additive effect) in reversing, preventing or slowing functional impairment the subject.

### Example 6.4

### Assessment of Efficacy of Laquinimod in Combination with Fingolimod in ALS Patients

The combination therapy provides a synergistic effect, and allow for lower doses with reduced side effects.

Periodic administration of laquinimod (p.o. 0.1, 0.15, 0.2, 0.25, 0.3 or 0.6 mg/day) in combination with fingolimod (p.o. 0.05, 0.1, 0.15, 0.2, 0.25, 0.3, 0.4, or 0.5 mg/day) to a human patient afflicted with ALS provides increased efficacy (provides at least an additive effect or more than an additive effect) in treating the patient than when laquinimod is administered alone or when fingolimod is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when laquinimod or fingolimod is administered alone (at the same dose) in the following manner:
1. The combination therapy is more effective (provides an additive effect or more than an additive effect) in prolonging survival of ALS patients.
2. The combination therapy is more effective (provides an additive effect or more than an additive effect) in maintaining or improving the ALS Functional Rating Scale-Revised (ALSFRS-R) total score in the subject.
3. The combination therapy is more effective (provides an additive effect or more than an additive effect) in reversing, preventing or slowing motor neuron damage in the subject.
4. The combination therapy is more effective (provides an additive effect or more than an additive effect) in reversing, preventing or slowing functional impairment the subject.
5. The combination therapy is more effective (provides an additive effect or more than an additive effect) in reversing, preventing or slowing motor function impairment in HD patients.
6. The combination therapy is more effective (provides an additive effect or more than an additive effect) in reversing, preventing or slowing functional impairment the subject.

### Example 6.5

### Assessment of Efficacy of Laquinimod as Add-on Therapy to Fingolimod and Fingolimod as Add-on Therapy to Laquinimod in HD Patients

The add-on therapy provides a synergistic effect, and allow for lower doses with reduced side effects.

Periodic administration of laquinimod (p.o. 0.1, 0.15, 0.2, 0.25, 0.3 or 0.6 mg/day) as an add-on therapy for a human patient afflicted with HD who is already receiving fingolimod (p.o. 0.05, 0.1, 0.15, 0.2, 0.25, 0.3, 0.4, or 0.5 mg/day) provides a clinically meaningful advantage and is more effective (provides at least an additive effect or more than an additive effect) in treating the patient than when fingolimod is administered alone (at the same dose).

Periodic administration fingolimod (p.o. 0.05, 0.1, 0.15, 0.2, 0.25, 0.3, 0.4, or 0.5 mg/day) as an add-on therapy for a human patient afflicted with HD who is already receiving of laquinimod (p.o. 0.1, 0.15, 0.2, 0.25, 0.3 or 0.6 mg/day) provides a clinically meaningful advantage and is more effective (provides at least an additive effect or more than an additive effect) in treating the patient than when laquinimod is administered alone (at the same dose).

The add-on therapies also provides efficacy (provides at least an additive effect or more than an additive effect) in treating the patient without undue adverse side effects or affecting the safety of the treatment. As compared to when each agent is administered alone:
1. The add-on therapy is more effective (provides an additive effect or more than an additive effect) in maintaining or reducing the severity of chorea in Huntington's disease (e.g., as measured by Unified Huntington's Disease Rating Scale (UHDRS) Maximal Chorea score).
2. The add-on therapy is more effective (provides an additive effect or more than an additive effect) in maintaining or improving cognitive function in HD patients.
3. The add-on therapy is more effective (provides an additive effect or more than an additive effect) in reversing, preventing or slowing cognitive impairment in HD patients.
4. The add-on therapy is more effective (provides an additive effect or more than an additive effect) in reversing, preventing or slowing motor function impairment in HD patients.
5. The add-on therapy is more effective (provides an additive effect or more than an additive effect) in reversing, preventing or slowing behavioral impairment in HD patients.
6. The add-on therapy is more effective (provides an additive effect or more than an additive effect) in reversing, preventing or slowing functional impairment the subject.

### Example 6.6

### Assessment of Efficacy of Laquinimod in Combination with Fingolimod in HD Patients

The combination therapy provides a synergistic effect, and allow for lower doses with reduced side effects.

Periodic administration of laquinimod (p.o. 0.1, 0.15, 0.2, 0.25, 0.3 or 0.6 mg/day) in combination with fingolimod (p.o. 0.05, 0.1, 0.15, 0.2, 0.25, 0.3, 0.4, or 0.5 mg/day) to a human patient afflicted with HD provides increased efficacy (provides at least an additive effect or more than an additive effect) in treating the patient than when laquinimod is administered alone or when fingolimod is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when laquinimod or fingolimod is administered alone (at the same dose) in the following manner:
1. The combination therapy is more effective (provides an additive effect or more than an additive effect) in maintaining or reducing the severity of chorea in Huntington's disease (e.g., as measured by Unified Huntington's Disease Rating Scale (UHDRS) Maximal Chorea score).
2. The combination therapy is more effective (provides an additive effect or more than an additive effect) in maintaining or improving cognitive function in HD patients.
3. The combination therapy is more effective (provides an additive effect or more than an additive effect) in reversing, preventing or slowing cognitive impairment in HD patients.
4. The combination therapy is more effective (provides an additive effect or more than an additive effect) in reversing, preventing or slowing motor function impairment in HD patients.
5. The combination therapy is more effective (provides an additive effect or more than an additive effect) in reversing, preventing or slowing behavioral impairment in HD patients.
6. The combination therapy is more effective (provides an additive effect or more than an additive effect) in reversing, preventing or slowing functional impairment the subject.

### Example 6.7

### Assessment of Efficacy of Laquinimod as Add-on Therapy to Fingolimod and Fingolimod as Add-on Therapy to Laquinimod in PD Patients

The add-on therapy provides a synergistic effect, and allow for lower doses with reduced side effects.

Periodic administration of laquinimod (p.o. 0.1, 0.15, 0.2, 0.25, 0.3 or 0.6 mg/day) as an add-on therapy for a human patient afflicted with PD who is already receiving fingolimod (p.o. 0.05, 0.1, 0.15, 0.2, 0.25, 0.3, 0.4, or 0.5 mg/day) provides a clinically meaningful advantage and is more effective (provides at least an additive effect or more than an additive effect) in treating the patient than when fingolimod is administered alone (at the same dose).

Periodic administration fingolimod (p.o. 0.05, 0.1, 0.15, 0.2, 0.25, 0.3, 0.4, or 0.5 mg/day) as an add-on therapy for a human patient afflicted with PD who is already receiving of laquinimod (p.o. 0.1, 0.15, 0.2, 0.25, 0.3 or 0.6 mg/day) provides a clinically meaningful advantage and is more effective (provides at least an additive effect or more than an additive effect) in treating the patient than when laquinimod is administered alone (at the same dose).

The add-on therapies also provides efficacy (provides at least an additive effect or more than an additive effect) in treating the patient without undue adverse side effects or affecting the safety of the treatment. As compared to when each agent is administered alone:
1. The add-on therapy is more effective (provides an additive effect or more than an additive effect) in maintaining or improving the Unified Parkinson's Disease Rating Scale (UPDRS) (Part III) Motor Score of the subject.
2. The add-on therapy is more effective (provides an additive effect or more than an additive effect) in maintaining or improving the Total Unified Parkinson's Disease Rating Scale (UPDRS) Score of the subject.
3. The add-on therapy is more effective (provides an additive effect or more than an additive effect) in maintaining or improving cognitive function in PD patients.
4. The add-on therapy is more effective (provides an additive effect or more than an additive effect) in reversing, preventing or slowing cognitive impairment in PD patients.
5. The add-on therapy is more effective (provides an additive effect or more than an additive effect) in reversing, preventing or slowing functional impairment in PD patients.

### Example 6.8

### Assessment of Efficacy of Laquinimod in Combination with Fingolimod in PD Patients

The combination therapy provides a synergistic effect, and allow for lower doses with reduced side effects.

Periodic administration of laquinimod (p.o. 0.1, 0.15, 0.2, 0.25, 0.3 or 0.6 mg/day) in combination with fingolimod (0.05, 0.1, 0.15, 0.2, 0.25, 0.3, 0.4, or 0.5 mg/day) to a human patient afflicted with PD provides increased efficacy (provides at least an additive effect or more than an additive effect) in treating the patient than when laquinimod is administered alone or when fingolimod is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when laquinimod or fingolimod is administered alone (at the same dose) in the following manner:
1. The combination therapy is more effective (provides an additive effect or more than an additive effect) in maintaining or improving the Unified Parkinson's Disease Rating Scale (UPDRS) (Part III) Motor Score of the subject.
2. The combination therapy is more effective (provides an additive effect or more than an additive effect) in maintaining or improving the Total Unified Parkinson's Disease Rating Scale (UPDRS) Score of the subject.
3. The combination therapy is more effective (provides an additive effect or more than an additive effect) in maintaining or improving cognitive function in PD patients.
4. The combination therapy is more effective (provides an additive effect or more than an additive effect) in reversing, preventing or slowing cognitive impairment in PD patients.
5. The combination therapy is more effective (provides an additive effect or more than an additive effect) in reversing, preventing or slowing functional impairment in PD patients.

### Example 7

### Evaluation of Efficacy of Laquinimod and Fingolimod in Combination on Inflammatory Cytokine Secretion and Neuronal Survival in Culture

### Experimental Protocol

### 1. Cortical Neurons Cell Culture

Mice cortical neurons were cultured as described by Singer et al., 1999. Briefly pregnant female mice of 13 days gestation were killed by cervical dislocation (Mice Swiss; Janvier Lab). The foetuses were removed from the uterus. The cortexes were removed and placed in ice-cold medium of Leibovitz (L15, Panbiotech, ref: P04-27055, batch: 9310614) containing 2% of Penicillin 10.000 U/ml and Streptomycin 10 mg/ml (PS, Panbiotech, ref: P06-07100, batch: 8460514) and 1% of Bovine Serum Albumin (BSA, Panbiotech, Ref: P06-1391100, batch: H140603). Cortexes were dissociated by trypsin-EDTA (Panbiotech, Ref: P10-023100, batch: 3330914) for 20 min at 37° C. The reaction was stopped by the addition of Dulbecco's modified Eagle's Medium (DMEM, Panbiotech, Ref P04-03600, batch: 1300714) containing DNasel grade II (0.1 mg/ml, Panbiotech, ref: P60-37780100, batch: H140508) and 10% of Foetal Calf Serum (FCS, Invitrogen, ref: 10270-098, batch: 4103912K). Cells were then mechanically dissociated by 3 serial passages through a 10 ml pipette. Cells were then centrifuged at 515×g for 10 min at 4° C. The supernatant was discarded and the pellet of cells was re-suspended in a defined culture medium consisting of Neurobasal (Nb, Invitrogen, ref: 21103, batch: 1673148) supplemented with B27 (2%, Invitrogen, ref: 17504, batch: 1672731), L-glutamine (2 mM, Panbiotech, ref: P04-80100, batch: 6620314), 2% of PS solution and 10 ng/ml of of Brain-derived neurotrophic factor (BDNF, PanBiotech, Ref: CB-1115002, Batch: 121027). Viable cells were counted in a Neubauer cytometer using the trypan blue exclusion test. The cells were seeded at a density of 30 000 cells/well in 96 well-plates pre-coated with poly-D-lysine (Greiner ref: 655930, batch: E140305F) and were cultured at +37° C. in a humidified air (95%)/CO2 (5%) atmosphere.

### 2. Preparation of Conditioned Media from Activated Astrocytes

### 2.1. Preparation and Purification of Astrocyte Culture

Mice mixed glial cells were cultured as described by McCarthy et al., 1980. Primary mice glial cells were prepared from the cortical of newborn Swiss mice (1 day). Briefly, meninges and blood vessels of the mice cortex were removed and placed in ice-cold medium of L15 containing 2% of PS and 1% of BSA. Tissues were dissociated with 0.25% trypsin-EDTA at 37° C. for 10 min. Cells were then submitted to a supplementary incubation of 15 min at 37° C. in presence of deoxyribonuclease I (final concentration of 0.5 mg/mL). Cells were then pelleted (5 min at 1200 rpm) and trypsinization was stopped by adding DMEM supplemented with 10% FCS, 1 mM of Na/pyruvate (PanBiotech, ref: P04-43100, batch: 3470914) and 2% PS. Cells suspension was mechanically dissociated and filtered through 40 μm diameter nylon meshes (BD Falcon, Ref: 352340). The cells were collected by centrifugation at 1200 rpm/min for 10 min, re-suspended in culture medium and then plated in culture flasks (Dutscher, ref: 690175). Cells were seeded at a density of 1.25×105 cells/cm2 and cultured in 5% CO2 at 37° C. Medium was changed three times per week.

Purification of astrocytes cells was done as described by Kim et al., 2006. After 14 days, the flasks were shaken on a rotary shaker at 200 rpm for 3 h. The resulting cell suspension rich in microglia was removed. Cells remaining in the flasks from which microglia had been harvested correspond to astrocytes at a purity of about 90%. Astrocytes were cultured in DMEM supplemented with 10% FCS, 1 mM of Na/pyruvate and 2% at 5% CO2 and 37° C. in flasks of 25 cm2.

### 2.2. LPS/INF-Gamma Exposure and Drug Treatment

When reaching confluence, primary astroglial cells were first incubated for 2 hours with Fingolimod (1 nM, 10 nM) or Laquinimod (1 nM, 10 nM, 100 nM, 1 μM, 10 μM) alone or in co-incubation or control medium.

At the end of 2 hours treatment with test compounds, astrocyte culture was activated with serum-free DMEM containing LPS (100 ng/mL; Sigma, Serotype 026:B6, ref: L2654; batch: 123M4052V) and IFNγ (10 ng/mL; Peprotech; ref: 315-05; batch: 061398 L0513) for 6 hours (Kim and Lee, 2013; Shu et al., 2014; Gresa-Arribas et al., 2012) in absence or presence of test compounds.

### 2.3. Conditioned Media Preparation

To obtain LPS/INFγ free conditioned media (CM), after 6 hours, cells were washed twice with DMEM and medium was replaced with DMEM supplemented with B27 (2%), L-glutamine (2 mM), PS solution (2%). LCM was collected 24 hrs after. For control, cells were incubated with medium not containing LPS/INFγ.

The following conditions were done:
Fresh CM from astroglial cells after 6 hours of incubation with control medium
Fresh CM from astroglial cells after 6 hours of stimulation by LPS (100 ng/mL) and IFNγ (long/mL)
Fresh CM from astroglial cells after a pre-incubation of 2 hours with Fingolimod (1 nM, 10 nM,) and then treated 6 hours with LPS (100 ng/mL) and IFNγ (10 ng/mL) in presence of Fingolimod (1 nM, 10 nM)
Fresh CM from astroglial cells after a pre-incubation of 2 hours with Laquinimod (1 nM, 10 nM, 100 nM, 1 μM) and then treated 6 hours with LPS (100 ng/mL) and IFNγ (long/mL) in presence of Laquinimod (1 nM, 10 nM, 100 nM, 1 μM).
Fresh CM from astroglial cells after a pre-incubation of 2 hours with Fingolimod (1 nM)+Laquinimod (1 nM) and then treated 6 hours with LPS (100 ng/mL) and IFNγ (10 ng/mL) in presence of Fingolimod (1 nM)+Laquinimod (1 nM)
Fresh CM from astroglial cells after a pre-incubation of 2 hours with Fingolimod (10 nM)+Laquinimod (1 nM) and then treated 6 hours with LPS (100 ng/mL) and IFNγ (10 ng/mL) in presence of Fingolimod (10 nM)+Laquinimod (1 nM)
Fresh CM from astroglial cells after a pre-incubation of 2 hours with Fingolimod (1 nM)+Laquinimod (10 nM) and then treated 6 hours with LPS (100 ng/mL) and IFNγ (10 ng/mL) in presence of Fingolimod (1 nM)+Laquinimod (10 nM)
Fresh CM from astroglial cells after a pre-incubation of 2 hours with Fingolimod (10 nM)+Laquinimod (10 nM) and then treated 6 hours with LPS (100 ng/mL) and IFNγ (10 ng/mL) in presence of Fingolimod (10 nM)+Laquinimod (10 nM)

### 3. Test of Conditioned Media on Cortical Neurons

To test toxicity of cytokines from conditioned media, 100 μL of LCM was added per well of 96 wells plate containing cortical neuron cultures on day 11, and was incubated for 72 hours. 6 wells per condition were performed.

The following conditions were done,
Fresh CM from astroglial cells after 6 hours of incubation with control medium; incubated with neuron during 72 hours
Control medium; incubated with neuron during 72 hours
Fresh CM from astroglial cells after 6 hours of stimulation by LPS (100 ng/mL) and IFNγ (10 ng/mL); incubated with neuron during 72 hours
Fresh CM from astroglial cells after a pre-incubation of 2 hours with Fingolimod (1 nM, 10 nM) and a 6 hours stimulation by LPS (100 ng/mL) and IFNγ (10 ng/mL) in presence of Fingolimod; incubated with neuron during 72 hours
Fresh CM from astroglial cells after a pre-incubation of 2 hours with Laquinimod (1 nM, 10 nM, 100 nM, 1 μM) and a 6 hours stimulation by LPS (100 ng/mL) and IFNγ (10 ng/mL) in presence of Laquinimod; incubated with neuron during 72 hours
Fresh CM from astroglial cells after a pre-incubation of 2 hours with Fingolimod (1 nM)+Laquinimod (1 nM) and a 6 hours stimulation by LPS (100 ng/mL) and IFNγ (10 ng/mL) in presence of Fingolimod and Laquinimod; incubated with neuron during 72 hours
Fresh CM from astroglial cells after a pre-incubation of 2 hours with Fingolimod (10 nM)+Laquinimod (1 nM) and a 6 hours stimulation by LPS (100 ng/mL) and IFNγ (10 ng/mL) in presence of Fingolimod and Laquinimod; incubated with neuron during 72 hours
Fresh CM from astroglial cells after a pre-incubation of 2 hours with Fingolimod (1 nM)+Laquinimod (10 nM) and a 6 hours stimulation by LPS (100 ng/mL) and IFNγ (10 ng/mL) in presence of Fingolimod and Laquinimod; incubated with neuron during 72 hours
Fresh CM from astroglial cells after a pre-incubation of 2 hours with Fingolimod (10 nM)+Laquinimod (10 nM) and a 6 hours stimulation by LPS (100 ng/mL) and IFNγ (long/mL) in presence of Fingolimod and Laquinimod; incubated with neuron during 72 hours

### 4. End Point Evaluation

### 4.1. Measure of Cytokine Content in CM

CM, prepared as described in section 2.3 was tested for the following cytokine levels:
1. TNF-alpha (BD Bioscience, ref: 562336; batch)
2. IL12 (BD Bioscience, ref: 558303, batch: 5036802)
3. IL6 (BD Bioscience, ref: 558301, batch: 4318913)
4. GM-CSF (BD Bioscience, ref: 558347, batch: 4197863)

Release in the media was quantified by flux cytometry with a CBA Mouse Soluble Protein Master Kit. 1500 events were recorded for each cytokine analysis in 1 lecture. Another lecture of the same sample isn't necessary with this cytometry test.
CCL7 (antibodies.online, ref: ABIN1029305, batch: EDL2015070205) and nitric oxide (antibodies.online ref: ABIN773480, batch: 20150703) content were quantified by ELISA. Six wells per condition of the same sample were done

### 4.2. Measure of Cortical Neurons Total Number

After 72 hours of cortical neurons intoxication in presence of CM, medium or control medium, cells were washed twice in phosphate buffered saline (PBS, PanBiotech, ref: P04-36500, Batch: 1870415) and then fixed by a solution of paraformaldhyde 4% (Sigma, Ref: P-6148; Batch: SLBH4356V) for 20 min at room temperature. The cells were then permeabilized and non-specific sites were blocked with a solution of PBS containing 0.1% of saponin (Sigma Aldrich, ref: S7900, Batch: BCBJ8417V) and 1% of FCS for 15 min at room temperature. Then, cells were incubated for 2 hr with primary antibody with a mouse monoclonal primary antibody anti-MAP2 (1/400, Sigma, ref: M4403 batch 063M4802) in PBS containing 1% FCS, 0.1% saponin. This antibody was revealed with Alexa Fluor 488 goat anti-mouse (Molecular probe, ref: A11001, Batch: 1572559) at 1/400 for 1 hr. Nuclei of cells were labeled by a fluorescent marker (Hoechst solution, SIGMA, ref: B1155, Batch: 011M4004V).

Six wells per condition (1 culture) were done to assess neuronal survival.

For each condition, 20 pictures per well were taken using using InCell Analyzer™ 2000 (GE Healthcare) with 20× magnification. All images were taken under the same conditions. Analysis of cortical cell bodies was performed using Developer software (GE Healthcare). A total of 6 data per experimental condition were provided.

### 8. Statistics

The data were expressed as mean±s.e. mean (6 per condition). A global analysis of the data was performed using unpaired t-test for ELISA and survival analysis; *p<0.05; **p<0.01; *** p<0.001. Effect of Laquinimod and Fingolimod combination in comparison to compounds alone was tested by a Bonferroni multiple comparisons tested *p<0.05; **p<0.01; *** p<0.001, **** p<0.0001.

### Results

According to FIG. 1, activation of purified astrocytes with INFγ (10 ng/mL) and LPS (100 ng/mL) led to a significant increase of NO release (***, p<0.001) from 7 pg/mL in control condition to 43 pg/mL in treated astrocytes. This result validated the study.

Laquinimod at all the concentrations tested was able to decrease the release of NO in a significant and dose dependent manner (***, p<0.001, respectively 26.44 pg/mL, 21.1 pg/mL, 17.98 pg/mL and 16.11 pg/mL).

Fingolimod was also able to decrease the release of NO in a significant and dose dependent manner at 1 nM and 10 nM (*** p<0.001, 24.39 pg/mL and 17.37 pg/mL respectively).

Laquinimod and Fingolimod when applied together were more effective than when they are applied alone (for example Laquinimod 10 nM+Fingolimod 10 nM vs Laquinimod 10 nM alone tested by a Bonferroni multiple comparison, ****, p<0.0001).

The effect of Laquinimod, Fingolimod, and a combination of Laquinimod and Fingolimod in decreasing the release of NO in treated astrocytes is shown below in Table 1.

TABLE 1

| Treatment | NO content (pg/mL) | % inhibition |
|---|---|---|
| L + I Stimulated | 43.34 | N.A. |
| supernatant | | |
| Laquinimod 1 nM | 26.447 | 39.0 |
| Laquinimod 10 nM | 21.093 | 51.3 |
| Laquinimod 100 nM | 17.983 | 58.5 |
| Laquinimod 1 μM | 16.111 | 62.8 |
| Fingolimod 1 nM | 24.396 | 43.7 |
| Fingolimod 10 nM | 17.371 | 59.9 |
| Laquinimod 1 nM + | 15.593 | 64.0 |
| Fingolimod 1 nM | | |
| Laquinimod 10 nM + | 6.788 | 84.3 |
| Fingolimod 10 nM | | |
| Laquinimod 1 nM + | 9.907 | 77.1 |
| Fingolimod 10 nM | | |
| Laquinimod 10 nM + | 7.844 | 81.9 |
| Fingolimod 1 nM | | |

According to FIG. 2, activation of purified astrocytes with INFγ (10 ng/mL) and LPS (100 ng/mL) led to a large increase of CCL7 release (***, p<0.001) from 17.79 pg/mL in control condition to 955 pg/mL in treated astrocytes. This result validated the study.

Laquinimod at 100 nM (*, p<0.05), 111M (**, p<0.01) was able to decrease the release of CCL7 in a significant manner (respectively 701 pg/mL, 664 pg/mL).

Fingolimod at 10 nM was able to decrease the release of CCL7 in a significant manner (*, p<0.05, 708 pg/mL). This effect was higher when Fingolimod 10 nM was applied with 10 nM Laquinimod (**, p<0.01; 614 ng/mL) but this difference was not significant (ns, p>0.05, Bonferroni multiple comparison).

Then, administration of Fingolimod and Laquinimod in combination was not more effective on the release of CCL7 than that observed when they were applied alone.

The effect of Laquinimod, Fingolimod, and a combination of Laquinimod and Fingolimod in decreasing the release of CCL7 in treated astrocytes is shown below in Table 2.

TABLE 2

| Treatment | CCL-7 content (pg/mL) | % inhibition |
|---|---|---|
| L + I Stimulated | 43.34 | N.A. |
| supernatant | | |
| Laquinimod 1 nM | 26.447 | 39.0 |
| Laquinimod 10 nM | 21.093 | 51.3 |
| Laquinimod 100 nM | 17.983 | 58.5 |
| Laquinimod 1 μM | 16.111 | 62.8 |
| Fingolimod 1 nM | 24.396 | 43.7 |
| Fingolimod 10 nM | 17.371 | 59.9 |
| Laquinimod 1 nM + | 15.593 | 64.0 |
| Fingolimod 1 nM | | |
| Laquinimod 10 nM + | 6.788 | 84.3 |
| Fingolimod 10 nM | | |
| Laquinimod 1 nM + | 9.907 | 77.1 |
| Fingolimod 10 nM | | |
| Laquinimod 10 nM + | 7.844 | 81.9 |
| Fingolimod 1 nM | | |

According to FIG. 3, stimulation of astrocytes with LPS (100 ng/mL) and IFNγ (10 ng/mL) led to a strong release of IL-6 in the supernatant (***, p<0.001) from 0.25 pg/mL in control condition to 7065 pg/mL in conditioned media from treated astrocytes.

Laquinimod showed a strong and significant inhibitory effect on IL-6 release at all the concentrations tested. The highest effect was seen at 1 μM (***, p<0.001, 2640 pg/mL). Effect of Laquinimod at 1 nM (6198 pg/mL) and 10 nM (6315 pg/mL) was a little bit higher when applied in combination with Fingolimod at 1 nM (****, p<0.0001, 5536.85 pg/mL and 5592/mL respectively, Bonferroni multiple comparison).

Fingolimod showed also a significant inhibitory effect on IL-6 release at 1 nM (***, p<0.001, 5421 pg/mL) and 10 nM (***, p<0.001, 4744 pg/mL). Effect of Fingolimod on IL-6 release was similar or weaker when applied with Laquinimod at 1 nM or 10 nM.

The effect of Laquinimod, Fingolimod, and a combination of Laquinimod and Fingolimod in inhibiting IL-6 release in treated astrocytes is shown below in Table 3.

TABLE 3

| Treatment | IL-6 content (pg/mL) | % inhibition |
|---|---|---|
| L + I Stimulated | 7065 | N.A. |
| supernatant | | |
| Laquinimod 1 nM | 6198 | 12.3 |
| Laquinimod 10 nM | 6315 | 10.6 |
| Laquinimod 100 nM | | |
| Laquinimod 1 μM | 2640 | 62.6 |
| Fingolimod 1 nM | 5421 | 23.3 |
| Fingolimod 10 nM | 4744 | 32.9 |
| Laquinimod 1 nM + | 5536 | 21.6 |
| Fingolimod 1 nM | | |
| Laquinimod 10 nM + | | |
| Fingolimod | | |
| 10 nM | | |
| Laquinimod 1 nM + | | |
| Fingolimod 10 nM | | |
| Laquinimod 10 nM + | 5592 | 20.8 |
| Fingolimod 1 nM | | |

As observed on FIG. 4, IL-12p70 release by astrocytes after their stimulation by LPS (100 ng/mL) and IFNγ (10 ng/mL) was higher than in control condition (***, p<0.001) but weak (9.94 pg/mL vs 0 pg/mL in control).

The release of IL12p70 was significantly blocked by Laquinimod at all the concentrations tested and this effect was dose dependent. The highest effect was seen at 1 μM (***, p<0.001; 2.55 pg/mL). Effect of Laquinimod at 1 nM (6.53 pg/mL) was stronger when applied in combination with Fingolimod at 10 nM (2.09 pg/mL)

Fingolimod showed also a Significant and dose dependent inhibitory effect at 1 nM (**, p<0.001, 6.15 pg/mL) and 10 nM, (***, p<0.001, 4.25 pg/mL). Effect of Fingolimod was not significantly different when co-incubated with Laquinimod (ns, p>0.05).

The effect of Laquinimod, Fingolimod, and a combination of Laquinimod and Fingolimod in blocking the release of IL12p70 in treated astrocytes is shown below in Table 4.

TABLE 4

| | IL-12p70 content | |
|---|---|---|
| Treatment | (pg/mL) | % inhibition |
| L + I Stimulated | 9.94 | N.A. |
| supernatant | | |
| Laquinimod 1 nM | 6.53 | 34.3 |
| Laquinimod 10 nM | 6.29 | 36.7 |
| Laquinimod 100 nM | 3.84 | 61.4 |
| Laquinimod 1 μM | 2.55 | 74.3 |
| Fingolimod 1 nM | 6.15 | 38.1 |
| Fingolimod 10 nM | 4.25 | 57.2 |
| Laquinimod 1 nM + | 4.99 | 49.8 |
| Fingolimod 1 nM | | |
| Laquinimod 10 nM + | 5.76 | 42.1 |
| Fingolimod | | |
| 10 nM | | |
| Laquinimod 1 nM + | | |
| Fingolimod 10 nM | | |
| Laquinimod 10 nM + | 5.38 | 45.9 |
| Fingolimod 1 nM | | |

According to FIG. 5, stimulation of astrocytes with LPS (100 ng/mL) and IFNγ (10 ng/mL) led to a strong release of TNFα in the supernatant (***, p<0.001) from 50.59 pg/mL in control condition to 3280 pg/mL in conditioned media from treated astrocytes.

Laquinimod showed a significant inhibitory effect on TNFα release at 10, 100 nM and 1 μM. The highest effect was seen at 100 nM (***, p<0.001, 2464 pg/mL) then regressed a little bit but stayed highly significant at 1 μM (***, p<0.001, 2769 pg/mL).

In contrast, Fingolimod didn't show any significant effect at 1 nM and 10 nM. Effect of Fingolimod at 10 nM was significantly higher when applied in combination with Laquinimod (Fingolimod 10 nM vs Laq 10 nM+Fingo 10 nM, *, p<0.05 tested by a Bonferroni multiple comparison).

Effect of Laquinimod was higher than effect of Fingolimod and was not better when applied in combination.

The effect of Laquinimod, Fingolimod, and a combination of Laquinimod and Fingolimod in inhibiting TNFα release in treated astrocytes is shown below in Table 5.

TABLE 5

| Treatment | TNFa content (pg/mL) | % inhibition |
|---|---|---|
| L + I Stimulated | 3280.21 | N.A. |
| supernatant | | |
| Laquinimod 1 nM | 3257.64 | 0.7 |
| Laquinimod 10 nM | 2440.81 | 25.6 |
| Laquinimod 100 nM | 2464 | 24.9 |
| Laquinimod 1 μM | 2769.46 | 15.6 |
| Fingolimod 1 nM | 3170.03 | 3.4 |
| Fingolimod 10 nM | 3053.66 | 6.9 |
| Laquinimod 1 nM + | 3184.67 | 2.9 |
| Fingolimod 1 nM | | |
| Laquinimod 10 nM + | 2787.69 | 15.0 |
| Fingolimod 10 nM | | |
| Laquinimod 1 nM + | 2611.1 | 20.4 |
| Fingolimod 10 nM | | |
| Laquinimod 10 nM + | 3157.61 | 3.7 |
| Fingolimod 1 nM | | |

According to FIG. 6, stimulation of astrocytes with LPS (100 ng/mL) and IFNγ (10 ng/mL) led to a significant release of GM-CSF in the supernatant (from 1.65 pg/mL in control to 243 pg/mL in conditioned media from treated astrocytes).

Laquinimod showed a significant and dose dependent inhibitory effect on GM-CSF release at all the concentration tested. The highest effect was seen at 1 μM (***, p<0.001, 154 pg/mL).

Fingolimod showed also a significant inhibitory effect on GM-CSF release. This effect was dose dependent and was the strongest at 10 nM (***, p<0.001, 148 pg/L).

Laquinimod and Fingolimod when applied together were more effective than when they were applied alone (****, p<0.0001 tested by a Bonferroni multiple comparison) except for the condition with Laquinimod at 10 nM and Fingolimod 10 nM.

The effect of Laquinimod, Fingolimod, and a combination of Laquinimod and Fingolimod in inhibiting GM-CSF release in treated astrocytes is shown below in Table 6.

TABLE 6

| Treatment | GM-CSF content (pg/mL) | % inhibition |
|---|---|---|
| L + I Stimulated | 242.87 | N.A. |
| supernatant | | |
| Laquinimod 1 nM | 207.71 | 14.5 |
| Laquinimod 10 nM | 209.51 | 13.7* |
| Laquinimod 100 nM | 179.39 | 26.1 |
| Laquinimod 1 μM | 154.26 | 36.5 |
| Fingolimod 1 nM | 204.09 | 16.0* |
| Fingolimod 10 nM | 147.56 | 39.2 |
| Laquinimod 1 nM + | 177.72 | 26.8 |
| Fingolimod 1 nM | | |
| Laquinimod 10 nM + | 163.39 | 32.7 |
| Fingolimod 10 nM | | |
| Laquinimod 1 nM + | 138.58 | 42.9 |
| Fingolimod 10 nM | | |
| Laquinimod 10 nM + | 112.8 | 53.6* |
| Fingolimod 1 nM | | |

*Concentrations of Laquinimod and/or Fingolimod that showed a synergistic effect in inhibiting GM-CSF release in treated astrocytes.

According to FIG. 7, firstly control conditioned media had a similar effect to control medium (99% of control, p>0.05, ns) on cortical neuron survival that validated the study.

Then, stimulated supernatant with LPS at 100 ng/mL and IFNγ at 10 ng/mL induced a significant decrease of cortical neuron survival (66% of control, ***, p<0.001).

Laquinimod at 10 nM (* p<0.05), 100 nM (**, p<0.01) and 1 μM (***, p<0.001) was able to significantly decrease cell death induced by conditioned media (80%; 83% and 92% of the control respectively). Effect of Laquinimod at 1 nM (73% of the control) was stronger when applied in co-incubation with Fingolimod at 10 nM (***, p<0.001, 94% of the control).

Fingolimod at 1 nM (*, p<0.05) and 10 nM (***, p<0.001) was also able to significantly decrease cell death induced by conditioned media (80% and 85% of the control respectively. The effect of Fingolimod was similar than that observed when applied in combination with Laquinimod (ns, p>0.05).

The effect of Laquinimod, Fingolimod, and a combination of Laquinimod and Fingolimod in decreasing cortical neuron cell death in treated astrocytes is shown below in Table 7.

TABLE 7

| Treatment | % cell survival | % cell death | % inhibition |
|---|---|---|---|
| L + I Stimulated | 66 | 34 | N.A. |
| supernatant | | | |
| Laquinimod 1 nM | 73 | 27 | 20.6* |
| Laquinimod 10 nM | 80 | 20 | 41.2 |
| Laquinimod | 83 | 17 | 50.0 |
| 100 nM | | | |
| Laquinimod 1 μM | 92 | 8 | 76.5 |
| Fingolimod 1 nM | 80 | 20 | 41.2 |
| Fingolimod 10 nM | 85 | 15 | 55.9* |
| Laquinimod 1 nM + | 81 | 19 | 44.1 |
| Fingolimod | | | |
| 1 nM | | | |
| Laquinimod 10 nM + | 85 | 15 | 55.9 |
| Fingolimod | | | |
| 10 nM | | | |
| Laquinimod 1 nM + | 94 | 6 | 82.4* |
| Fingolimod | | | |
| 10 nM | | | |
| Laquinimod 10 nM + | 88 | 12 | 64.7 |
| Fingolimod | | | |
| 1 nM | | | |

*Concentrations of Laquinimod and/or Fingolimod that showed a synergistic effect in decreasing cortical neuron cell death in treated astrocytes.

### CONCLUSION

Laquinimod and Fingolimod were able to decrease the release of NO at all the concentration tested. Their effect was stronger when they were applied in combination. The strongest effect was seen with the combination Laquinimod at 10 nM and Fingolimod at 10 nM.

Laquinimod (at 100 nM and 1 μM) and Fingolimod (at 10 nM) were able to decrease the release of CCL7. Their effect was similar when they were applied in combination. The strongest effect was seen with the combination Laquinimod at 10 nM and Fingolimod at 10 nM but this effect was not significantly different from Fingolimod at 10 nM alone.

Laquinimod and Fingolimod were able to decrease the release of IL-6 at all the concentrations tested. At the same concentration, effect of Fingolimod seemed to be stronger than effect of Laquinimod and co-incubation with the two compounds did not give a better effect.

Laquinimod and Fingolimod were able to decrease the release of IL12p70 at all the concentrations tested. Their effect was not significantly different when they were applied in combination but the strongest effect was seen with the combination Laquinimod at 1 nM and Fingolimod at 10 nM.

Laquinimod but not Fingolimod was able to decrease the release of TNFα. At the same concentration, effect of Laquinimod was stronger than effect of Fingolimod and co-incubation with the two compounds did not give a better effect.

Laquinimod and Fingolimod were able to decrease the release of GM-CSF at all the concentrations tested. Their effect was stronger when they were applied in combination except when they were administrated both at 10 nM. The strongest effect was seen with the combination Laquinimod at 1 nM and Fingolimod at 10 nM.

Laquinimod (10 nM, 100 nM and 1 μM) and Fingolimod (1 nM and 10 nM) were able to significantly rescue neurons from the cell death induced by the conditioned media from reactive astrocytes.

The highest effect was seen with the combination of Laquinimod at 1 nM and Fingolimod at 10 nM.

### REFERENCES

1. “FDA approves first oral drug to reduce MS relapses” FDA NEWS RELEASE, Sep. 22, 2010.
2. Anderson, Pauline (2003) “Multiple Sclerosis: Autoimmune or Neurodegenerative?” 5th Cooperative Meeting of the Consortium of Multiple Sclerosis Centers (CMSC) and the Americas Committee for Treatment and Research In Multiple Sclerosis (ACTRIMS), May 29-Jun. 1, 2013; Orlando, Fla. (Medscape Medical News, Jun. 4, 2013).
3. Animal Models for Neurodegenerative Disease (2011), Editor(s): Jesus Avila, Jose J Lucas, Felix Hernandez, Royal Society of Chemistry, ISBN: 978-1-84973-184-3.
4. Archer S. (1993). “Measurement of nitric oxide in biological models”, FASEB 1, 7:349.
5. Berdyshev et al. (2009). “FTY720 inhibits ceramide synthases and up-regulates dihydrosphingosine 1-phosphate formation in human lung endothelial cells,” Journal of Biological Chemistry 284 (9): 5467-77.
6. Bertram and Tanzi (2005) “The genetic epidemiology of neurodegenerative disease,” J Clin Investig. 115:1449-57.
7. Billich et al. (2003). “Phosphorylation of the immunomodulatory drug FTY720 by sphingosine kinases”. J Biol Chem 278 (48): 47408-15.
8. Brod et al. (2000) Annals of Neurology, 47:127-131.
9. Brück (2011) “Insight into the mechanism of laquinimod action.” J Neurol Sci. 2011 Jul. 15; 306(1-2):173-9.
10. Brück and Zamvil (2012) Expert Rev. Clin. Pharmacol. 2012; 5(3), 245-256.
11. Brück et al. (2012) Acta neuropathologica 124:411-424.
12. Chesselet, M F (2003) “Dopamine and Parkinson's disease: is the killer in the house?” Molecular Psychiatry, 8:369-370.
13. Ciammola, A, et al. (2007) “Low brain-derived neurotrophic factor (BDNF) levels in serum of Huntington's disease patients”. Am J Med Gent Part 8, 144b:574-577.
14. Comi et al. (2007) LAQ/5062 Study Group. “The Effect of Two Doses of Laquinimod on MRI-Monitored Disease Activity in Patients with Relapsing-Remitting Multiple Sclerosis: A Multi-Center, Randomized, Double-Blind, Placebo-Controlled Study”, Presented at: 59th Annual Meeting of the American Academy of Neurology; Apr. 28-May 5, 2007; Boston, Mass.
15. Conway and Cohen (2010) “Combination therapy in multiple sclerosis”, LancetNeurol, 9:299-308.
16. Costello et al. (2007) “Combination therapies for multiple sclerosis: scientific rationale, clinical trials, and clinical practice”, Current Opinion in Neurology, 20:281-285.
17. Di Menna et al. (2013) Pharmacology res.67:1-9.
18. EMEA Guideline on Clinical Investigation of Medicinal Products for the Treatment of Multiple Sclerosis (CPMP/EWP/561/98 Rev. 1, November 2006).
19. Fernández (2007) “Combination therapy in multiple sclerosis”, Journal of the neurological sciences, 259:95-103.
20. Foster et al. (2007) JPET 323:469-476.
21. Frenández (2007) “Combination therapy in multiple sclerosis”, Journal of the neurological sciences, 259:95-103.
22. Gold (2008) “Combination therapies in multiple sclerosis”, J Neurol, 255[Suppl 1]:51-60.
23. Gresa-Arribas N., Viéitez C., Dentesano G., Serratosa J., Saura J., Solà., (2012) “Modelling Neuroinflammation In Vitro: A Tool to Test the Potential Neuroprotective Effect of Anti Inflammatory Agents”, Plos One, 7(9):e45227.
24. Griffith W. and Stueh D. (1995). “Nitric oxide synthase: Properties and Catalytic Mechanism”, Annual Rev. Physiol., 57:707-36.
25. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin. Pharm., November 1999 <http://www.fda.gov/cber/gdlns/metabol.pdf>.
26. Gurevich et al. (2010) “Laquinimod suppress antigen presentation in relapsing-remitting multiple sclerosis: in vitro high-throughput gene expression study” (J Neuroimmunol. 2010 Apr. 15; 221(1-2):87-94. Epub 2010 Mar. 27.
27. Hafler and Weiner, M S: A CNS and systemic autoimmune disease, Immunol. Today, 1989, 10:104-107.
28. Hla T, Lee M J, Ancellin N, Paik J H, Kluk M J (2001). “Lysophospholipids—receptor revelations”. Science 294 (5548): 1875-8.
29. Horga, Alejandro and Montalban, Xavier. Jun. 4, 2008; Expert Rev Neurother. 2008; 8(5):699-714.
30. Howells, D W, et al. (2000) “Reduced BDNF mRNA expression in the Parkinson's disease substantia nigra”. Experimental Neurology, 166(1):127-135.
31. Hu, Yinghui et al. (2001) Mol. Cell. Neuroscience, 48:72-81.
32. Hyman, C. et al., (1991) “BDNR is a neurotrophic factor for dopaminergic neurons of the substantia nigra”. Nature, 350(6315):230-2.
33. Katoh-Semba, R, et al. (2002) “Riluzole enhances expression of brain-derived neurotrophic factor with consequent proliferation of granule precursor cells in the rat hippocampus”. FASEB J, 16:1328-30.
34. Kim et al., (Jan. 19, 2011) “Neurobiological effects of sphingosine 1-phosphate” receptor modulation in the cuprizone model” The FASEB Journal. 25:1-10.
35. Kim I D. and Lee J K. (2013) HMGB1-Binding Heptamer Confers Anti-Inflammatory Effects in Primary Microglia Culture. Exp Neurol., 22(4), 301-307.
36. Kim J. B. et al. (2006) “HMGB1, a novel cytokine-like mediator linking acute neuronal death and delayed neuroinflammation in the postischemic brain”, J Neurosci, 26:6413-6421.
37. Kleinschmidt-DeMasters et al. (2005) New England Journal of Medicine, 353:369-379.
38. Lampert (1978) “Autoimmune and virus-induced demyelinating diseases. A review”, Am. J. Path., 91:176-208.
39. Langer-Gould et al. (2005) New England Journal of Medicine, 353:369-379.
40. McCarthy K D, de Vellis J. (1980) “Preparation of separate astroglial and oligodentroglial cell cultures from rat cerebral tissue”, J Cell Biol, 85(3), 890-902.
41. Milo and Panitch (2011) “Combination therapy in multiple sclerosis,” Journal of Neuroimmunology, 231(2011):23-31.
42. Paugh et al. (2003) “The immunosuppressant FTY720 is phosphorylated by sphingosine kinase type 2,” FEBS Lett 554 (1-2): 189-93.
43. Paugh et al. (2006) “Sphingosine and its analog, the immunosuppressant 2-amino-2-(2-[4-octylphenyl]ethyl)-1,3-propanediol, interact with the CB1 cannabinoid receptor,” Mol Pharmacol. 70 (1): 41-50.
44. Payne et al. (2007) “The immunosuppressant drug FTY720 inhibits cytosolic phospholipase A2 independently of sphingosine-1-phosphate receptors,” Blood 109 (3): 1077-85. doi:10.1182/blood-2006-03-011437.
45. PCT International Application Publication No. WO 1998/030227, published Jul. 16, 1998.
46. PCT International Application Publication No. WO 2000/005250, published Feb. 3, 2000.
47. PCT International Application Publication No. WO 2000/018794, published Apr. 6, 2000.
48. PCT International Application Publication No. WO 2003/048735, published Jun. 12, 2003.
49. PCT International Application Publication No. WO 2004/103297, published Dec. 2, 2004.
50. PCT International Application Publication No. WO 2006/0016036, published Nov. 2, 2006.
51. PCT International Application Publication No. WO 2006/029393, published Mar. 16, 2006.
52. PCT International Application Publication No. WO 2006/029411, published Mar. 16, 2006.
53. PCT International Application Publication No. WO 2006/083608, published Aug. 10, 2006.
54. PCT International Application Publication No. WO 2006/089164, published Aug. 24, 2006.
55. PCT International Application Publication No. WO 2006/116602, published Nov. 2, 2006.
56. PCT International Application Publication No. WO 2007/0047863, published Apr. 26, 2007.
57. PCT International Application Publication No. WO 2007/0146248, published Dec. 21, 2007.
58. PCT International Application Publication No. WO 2009/070298, published Jun. 4, 2009.
59. PCT International Application Publication No. WO 2011/008274, published Jan. 20, 2011.
60. PCT International Application Publication No. WO 2011/022063, published Feb. 24, 2011.
61. PCT International Application Publication No. WO 2012/0051106, published Apr. 19, 2012.
62. Pelletier and Hafler (2012) “Fingolimod for Multiple Sclerosis” New England Journal of Medicine, 366(4):339-347.
63. Phillips et al. (2009) “Animal Models of Neurodegenerative Diseases” Methods in Molecular Biology, 549:137-155.
64. Polman et al., (2005) “Treatment with laquinimod reduces development of active MRI lesions in relapsing MS”, Neurology. 64:987-991.
65. Ramaswamy et al. (2007) “Animal models of Huntington's disease,” ILAR J. 2007; 48(4):356-73.
66. Riviere, M (1998) “An analysis of extended survival in patients with amyotrophic lateral sclerosis treated with riluzole,” Arch Neurol, 55:526-8.
67. Rochelle et al. (2007) J. Pharmacology and Experimental Therapeutics 323(2):626-635.
68. RTT News Article dated Apr. 12, 2011, entitled “Teva Pharma, Active Biotech Post Positive Laquinimod Phase 3 ALLEGRO Results”.
69. Rudick et al. (2006) New England Journal of Medicine, 354:911-923.
70. Rudick, R. (1999) “Disease-Modifying Drugs for Relapsing-Remitting Multiple Sclerosis and Future Directions for Multiple Sclerosis Therapeutics,” Neurotherpatueics. 56:1079-1084.
71. Runström et al. (2002) “Laquinimod (ABR-215062) a candidate drug for treatment of Multiple Sclerosis inhibits the development of experimental autoimmune encephalomyelitis in IFN-β knock-out mice,” (Abstract), Medicon Valley Academy, Malmoe, Sweden.
72. Runstrom et al. (2006) “Inhibition of the development of chronic experimental autoimmune encephalomyelitis by laquinimod (ABR-215062) in IFN-β k.o. and wild type mice,” Journal of Neuroimmunology, 173(2006):69-78.
73. Sanchez et al. (2003) “Phosphorylation and action of the immunomodulator FTY720 inhibits vascular endothelial cell growth factor-induced vascular permeability,” The Journal of biological chemistry 278 (47): 47281-90.
74. Sandberg-Wollheim et al. (2005) “48-week open safety study with high-dose oral laquinimod in patients,” Mult Scler. 11:5154 (Abstract).
75. Schinelli, S. et al. (1988) “1-Methyl-4-phenyl-1,2,3,6-tetrahydropyridine metabolism and 1-methyl-4-phenylpyridinium uptake in dissociated cell cultures from the embryonic mesencephalon”, J Neurochem., 50, 1900-1907.
76. Shu Z. et al. (2014) “Tangeretin exerts anti-neuroinflammatory effects via NF-κB modulation in lipopolysaccharide-stimulated microglial cells”, Int Immunopharmacol., 19(2):275-82.
77. Singer C. et al. (1999) “Mitogen-activated protein kinase pathway mediates estrogen neuroprotection after glutamate toxicity in primary cortical neurons”, J. Neuroscience, 19(7):2455-2463
78. Slovic et al. ECTRIMS poster number 511, 2012.
79. Teva Press Release dated Aug. 1, 2011, entitled “Results of Phase III BRAVO Trial Reinforce Unique Profile of Laquinimod for Multiple Sclerosis Treatment”.
80. The Merck Manual of Diagnosis and Therapy, Seventeenth Edition, Ed. Mark H. Beers, M. D., and Robert Berkow, M. D., Merck Research Laboratories, Whitehouse Station, N J, 1999.
81. U.S. Patent Application Publication No. 2008-0207526, published Aug. 28, 2008 (Strominger et al.).
82. U.S. Patent Application Publication No. 2009-0081237, published Mar. 26, 2009 (D'Andrea et al.).
83. U.S. Patent Application Publication No. 2009-0176744, published Jul. 9, 2009 (Liu et al.).
84. U.S. Patent Application Publication No. 2010-0055072, published Mar. 4, 2010 (Gant et al.).
85. U.S. Patent Application Publication No. 2010-0322900, published Dec. 23, 2010 (Tarcic et al.).
86. U.S. Patent Application Publication No. 2011-0027219, published Feb. 3, 2011 (Tarcic et al.).
87. U.S. Patent Application Publication No. 2011-0034508, published Feb. 10, 2011 (Liat Hayardeny).
88. U.S. Patent Application Publication No. 2011-0152380, published Jun. 23, 2011 (Bastien et al.).
89. U.S. Patent Application Publication No. 2011-0217295, published Sep. 8, 2011 (Haviv and Tarcic).
90. U.S. Patent Application Publication No. 2011-0218179, published Sep. 8, 2011 (Haviv and Tarcic).
91. U.S. Patent Application Publication No. 2011-0218203, published Sep. 8, 2011 (Joel Kaye et al.).
92. U.S. Patent Application Publication No. 2011-0230413, published Sep. 22, 2011 (Suhayl Dhib-Jalbut).
93. U.S. Patent Application Publication No. 2012-0010238, published Jan. 12, 2012 (Fristedt).
94. U.S. Patent Application Publication No. 2012-0010239, published Jan. 12, 2012 (Piryatinsky et al.).
95. U.S. Patent Application Publication No. 2012-0142730, published Jun. 7, 2012 (Tarcic et al.).
96. U.S. Patent Application Publication No. 2012-0184617, published Jul. 19, 2012 (Gidwani et al.).
97. U.S. Pat. No. 3,849,550, issued Nov. 19, 1974 (Teitelbaum et al).
98. U.S. Pat. No. 5,719,176, issued Feb. 17, 1998 (Fujita et al).
99. U.S. Pat. No. 5,800,808, issued Sep. 1, 1998 (Konfino et al).
100. U.S. Pat. No. 5,858,964, issued Jan. 12, 1999 (Aharoni et al).
101. U.S. Pat. No. 5,981,589, issued Nov. 9, 1999 (Konfino et al).
102. U.S. Pat. No. 6,048,898, issued Apr. 11, 2000 (Konfino et al).
103. U.S. Pat. No. 6,054,430, issued Apr. 25, 2000 (Konfino et al).
104. U.S. Pat. No. 6,077,851, issued Jun. 20, 2000 (Bjork et al).
105. U.S. Pat. No. 6,214,791, issued Apr. 10, 2001 (Arnon et al).
106. U.S. Pat. No. 6,342,476, issued Jan. 29, 2002 (Konfino et al).
107. U.S. Pat. No. 6,362,161, issued Mar. 26, 2002 (Konfino et al).
108. U.S. Pat. No. 7,566,767, issued Jul. 28, 2009 (Strominger et al.).
109. U.S. Pat. No. 7,589,208, issued Sep. 15, 2009 (Jansson et al).
110. U.S. Pat. No. 7,884,208, issued Feb. 8, 2011 (Frenkel et al.).
111. U.S. Pat. No. 7,989,473, issued Aug. 2, 2011 (Patashnik et al.).
112. U.S. Pat. No. 8,008,258, issued Aug. 30, 2011 (Aharoni et al).
113. U.S. Pat. No. 8,178,127, issued May 15, 2012 (Safadi et al.).
114. Vollmer et al. (2008) “Glatiramer acetate after induction therapy with mitoxantrone in relapsing multiple sclerosis” Multiple Sclerosis, 00:1-8.
115. Webb et al. (2004) J. Neuroimmunol. 153:108-121.
116. Wegner et al. (2010) J. Neuroimmunol. 227(1-2):133-143.
117. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.
118. Yong (2002) “Differential mechanisms of action of interferon-β and glatiramer acetate in MS” Neurology, 59:1-7.
119. Amor et al. (2010) “Inflammation in neurodegenerative diseases”. Immunology, 129:154-169.
120. Barbierato (2012) “Astrocyte-microglia interaction in the expression of a pro-inflammatory or pain-related phenotype: molecular and cellular aspects” Thesis, Universita degli Studi di Padova, Dipartimentodi Farmacologia ed Anestesiologia.
121. Barreto et al. “Role of Astrocytes in Neurodegenerative Diseases” Chapter 11, Neurodegenerative diseases—Processes, Prevention, Protection and Monitoring. Edited by Raymond Chuen-Chung Chang, ISBN 978-953-307-485-6, 558 pages, Publisher: InTech, Chapters published Dec. 9, 2011 under CC BY 3.0 license.
122. Carson et al. (2006) “The cellular response in neuroinflammation: The role of leukocytes, microglia and astrocytes in neuronal death and survival”. Clin Neurosci Res. 2006 December; 6(5): 237-245.
123. Cerbai et al. (2012) “The neuron-astrocyte-microglia triad in normal brain ageing and in a model of neuroinflammation in the rat hippocampus”. PLoS ONE 7:e45250.
124. Giuliano (2011) “Key roles of glia and microglia in age-related neurodegenerative diseases” Aging Sciences—AntiAging Firewalls, retrieved from <www.anti-agingfirewalls.com/2011/11/03/key-roles-of-glia-and-microglia-in-age-related-neurodegenerative-disease/> on Dec. 17, 2013.
125. Liu and Hong (2003) “Role of Microglia in Inflammation—Mediate Neurodegenerative Diseases: Mechanisms and Strategies for Therapeutic Intervention” The Journal of Pharmacology and Experimental Therapeutics, 304:1-7.
126. Lobsiger and Cleveland (2007) “Glia cells as intrinsic components of non-cell autonomous neurodegenerative diseases” Nat Neurosci. 2007 November; 10(11):1355-1360.
127. Maragakis and Rothstein (2006) “Mechanisms of Diseases: astrocytes in neurodegenerative disease” Nature Clinical Practice Neurology, 2(12):679-689.
128. Mattson and Camandola (2001) “NF-κB in neuronal plasticity and neurodegenerative disorders” The Journal of Clinical Investigation, 107(3)247-254.
129. Taboada et al. “Microglia, Calcification and Neurodegenerative Diseases” Chapter 13, Neurodegenerative diseases—Processes, Prevention, Protection and Monitoring. Edited by Raymond Chuen-Chung Chang, ISBN 978-953-307-485-6, 558 pages, Publisher: InTech, Chapters published Dec. 9, 2011 under CC BY 3.0 license.

1. A method of treating a subject afflicted with a neurodegenerative disease comprising periodically administering to the subject an amount of laquinimod and an amount of fingolimod, wherein the amounts when taken together are effective to treat the subject.

2. The method of claim 1, wherein the amount of laquinimod and the amount of fingolimod when administered together is more effective to treat the subject than when each agent at the same amount is administered alone.

3. The method of claim 1, wherein
a) the neurodegenerative disease other than a form of multiple sclerosis,
b) the neurodegenerative disease is Alzheimer's disease, Amyotrophic lateral sclerosis, Huntington's disease, Parkinson's disease, Alexander disease, cerebellar ataxia, spinocerecellar ataxia (SCA), Batten disease, Creutzfeldt-Jakob disease, Charcot-Marie-Tooth disease (CMT), HIV-associated dementia, multiple system atrophy (MSA) or prion-related disease,
c) the neurodegenerative disease is a degenerative disease of the Central Nervous System (CNS), or
d) the neurodegenerative disease is a degenerative disease of the Peripheral Nervous System (PNS).

4-6. (canceled)

7. The method of claim 1, wherein the amount of laquinimod and the amount of fingolimod when taken together are effective to reduce a symptom of the neurodegenerative disease in the subject, wherein
a) if the disease is Alzheimer's disease, the symptom is dementia, memory loss, cognitive impairment, personality change, psychiatric disorder, or functional impairment,
b) if the disease is Amyotrophic lateral sclerosis and the symptom is cognitive impairment, motor function impairment, muscle disorder, fatigue, or functional impairment,
c) if the disease is Huntington's disease and the symptom is memory loss, psychiatric disorder, cognitive impairment, motor function impairment, chorea, seizure, or functional impairment, or
d) if the disease is Parkinson's disease and the symptom is dementia, bradyphrenia, psychiatric disorder, cognitive impairment, motor function impairment, tremor, rigidity, bradykinesia, postural dysfunction, or functional impairment.

8-11. (canceled)

12. The method of claim 1, wherein the amount of laquinimod and the amount of fingolimod when taken together are effective to reduce cellular production of pro-inflammatory mediator.

13. The method of claim 12, wherein the pro-inflammatory mediator is nitric oxide (NO), chemokine (C-C motif) ligand 7 (CCL-7), interleukin-6 (IL-6), interleukin-12p70 (IL-12p70), tumor necrosis factor alpha (TNF-α), and/or granulocyte-macrophage colony-stimulating factor (GM-CSF).

14-19. (canceled)

20. The method of claim 1, wherein the amount of laquinimod and the amount of fingolimod when taken together are effective to increase neuron survival and/or decrease neuron death.

21. The method of claim 20, wherein the neuron is cortical neuron.

22. The method of claim 1, wherein laquinimod is laquinimod sodium.

23. The method of claim 1, wherein fingolimod is fingolimod hydrochloride.

24. The method of claim 1, wherein the laquinimod and/or the fingolimod is administered via oral administration.

25. The method of claim 1, wherein the laquinimod and/or the fingolimod is administered daily, more often than once daily, or less often than once daily.

26. (canceled)

27. (canceled)

28. The method of claim 1, wherein the amount laquinimod administered is less than 0.6 mg/day, or is 0.1-40.0 mg/day, 0.1-2.5 mg/day, 0.25-2.0 mg/day, 0.5-1.2 mg/day, 0.25 mg/day, 0.3 mg/day, 0.5 mg/day, or 0.6 mg/day.

29-36. (canceled)

37. The method of claim 1, wherein the amount fingolimod administered is less than 0.5 mg/day, or is 0.01-2.5 mg/day, 0.01-1 mg/day, 0.1 mg/day, 0.25 mg/day, or 0.5 mg/day.

38-42. (canceled)

43. The method of claim 1, wherein a loading dose of an amount different from the intended dose is administered for a period of time at the start of the periodic administration.

44. (canceled)

45. The method of claim 1, wherein the subject is receiving laquinimod therapy prior to initiating fingolimod therapy, or wherein the subject is receiving fingolimod therapy prior to initiating laquinimod therapy.

46-50. (canceled)

51. The method of claim 1, further comprising administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

52. The method of claim 1, wherein the periodic administration of laquinimod and fingolimod continues for at least 3 days, more than 30 days, more than 42 days, 8 weeks or more, at least 12 weeks, at least 24 weeks, more than 24 weeks, or 6 months or more.

53-59. (canceled)

60. The method of claim 1, wherein each of the amount of laquinimod or pharmaceutically acceptable salt thereof when taken alone, and the amount of fingolimod when taken alone is effective to treat the subject, or wherein either the amount of laquinimod or pharmaceutically acceptable salt thereof when taken alone, the amount of fingolimod when taken alone, or each such amount when taken alone is not effective to treat the subject.

61. (canceled)

62. The method of claim 1, wherein the subject is a human patient.

63-161. (canceled)
